SEQUENCE LISTING (6 sequences)

SEQ ID NO: 1 - protein, 20 aa, Artificial sequence
  Synthetic construct
GWTLNSAGYLLGPPPALALA

SEQ ID NO: 2 - protein, 25 aa, Artificial sequence
  Synthetic construct
TTCACCATAGCCAGTTCCATCACTT

SEQ ID NO: 3 - protein, 23 aa, Artificial sequence
  Synthetic construct
AGTTGTGCCAGCCAGGGAAAACT

SEQ ID NO: 4 - protein, 57 aa, Artificial sequence
  Synthetic construct
CCGGCAAGTGTTCAAGTGTCACATTCTCGAGAATGTGACACTTGAACACTTGTTTTT

SEQ ID NO: 5 - protein, 22 aa, Artificial sequence
  Synthetic construct
WTLNSAGYLLGPEHPPPALALA

SEQ ID NO: 6 - protein, 24 aa, Artificial sequence
  Synthetic construct
TTGAGCATACCCACTTCGATCCTT

(19) United States
(12) Patent Application Publication
(10) Pub. No.: US 2022/0233640 A1
(43) Pub. Date: Jul. 28, 2022

(54) GALANIN- AND GALANIN RECEPTOR BASED COMPOUNDS FOR THE TREATMENT OF LIVER FIBROSIS

(71) Applicant: United State Govermment as represented by the Department of Veterans, Washington, DC (US)

(72) Inventors: Sharon Demorrow, Temple, TX (US); Matthew McMillin, Harker Heights, TX (US); Anca D. Petrescu, Temple, TX (US)

(21) Appl. No.: 17/604,370

(22) PCT Filed: Apr. 16, 2020
(86) PCT No.: PCT/US2020/028445

§ 371 Date: Oct. 15, 2021

Related U.S. Application Data
(60) Provisional application No. 62/835,166, filed on Apr. 17, 2019.

(51) Int. Cl. A61K 38/17; A61P 1/16; C12N 15/113

(52) U.S. Cl. CPC A61K 38/1709; A61P 1/16; C12N 2310/14; C12N 2310/3233; C12N 15/1138

(57) ABSTRACT

In one aspect, the invention relates to pharmaceutical compositions comprising at least one agent that modulates GalR1 and/or GalR, which are useful for treating fibrotic disorders such as, for example, liver fibrosis. This abstract is intended as a scanning tool for purposes of searching in the particular art and is not intended to be limiting of the present invention.

### CROSS-REFERENCE TO RELATED APPLICATIONS

This application claims the benefit of U.S. Application No. 62/835,166, filed on Apr. 17, 2019, the contents of which are hereby incorporated by reference in their entirety.

### STATEMENT REGARDING FEDERALLY SPONSORED RESEARCH

This invention was made with government support under grant numbers DK082435 and DK112803, awarded by the National Institutes of Health and grant numbers BX002638 and BX003486, awarded by the United States Department of Veterans Affairs Biomedical Laboratory Research and Development Service. The government has certain rights in the invention.

### REFERENCE TO SEQUENCE LISTING

The Sequence Listing submitted Apr. 16, 2020 as a text file named “37759_0213P1_ST25.txt,” created on Apr. 13, 2020, and having a size of 2,190 bytes is hereby incorporated by reference pursuant to 37 C.F.R. § 1.52(e)(5).

### BACKGROUND

Galanin (Gal) is a 29 amino acid neuropeptide, distributed throughout the central and peripheral nervous system, with high concentrations in amygdaloid nuclei, hypothalamus, locus coeruleus, and the sacral spinal cord (Ch′ng et al. (1985) Neuroscience 16: 343-54; Melander et al. (1986) Neuroscience 19: 223-40; Melander et al. (1986) Eur. J. Pharmacol. 124: 381-2; Skofitsch and Jacobowitz (1986) Peptides 7: 609-13). Central Gal was found to stimulate the release of hypothalamic vasoactive intestinal peptide (VIP) resulting in pituitary secretion of prolactin, and growth hormone (GH) via GH releasing hormone (GHRH) (Koshiyama et al. (1987) Neurosci. Lett. 75: 49-54; Murakami et al. (1987) Eur. J. Pharmacol. 136: 415-8; Inoue et al. (1988) Neurosci. Lett. 85: 95-100). Galanin has been shown to co-localize with several neuromodulators including GHRH, substance P, and VIP in the hypothalamus. Galanin is also expressed in the gastrointestinal tract, with highest concentration in the duodenum, and progressively lower abundance in the stomach, small intestine and colon (Kaplan et al. (1988) Proc. Natl. Acad. Sci. USA 85: 1065-9). Galanin was also identified in endocrine tissues such as anterior pituitary and adrenal glands, being characterized as a neuroendocrine peptide (Lang et al. (2015) Pharmacol. Rev. 67: 118-75).

Galanin regulates neuroendocrine signaling pathways which modulate food intake, and especially fat intake and metabolism (Yun et al. (2005) Peptides 26: 2265-73; Kyrkouli et al. (1990) Peptides 11: 995-1001). Galanin exerts its' actions through three types of receptors, known as GalR1, GalR2, and GalR3, which are G-protein coupled receptors (GPCR) with different distribution throughout the body (Wynick et al. (1993) Proc. Natl. Acad. Sci. USA 90: 4231-5; Parker et al. (1995) Brain Res. Mol. Brain Res. 34: 179-89; Howard et al. (1997) FEBS Lett. 405: 285-90; Wang et al. (1997) J. Biol. Chem. 272: 31949-52). Specifically, GalR1 is expressed in the basal forebrain, hypothalamus, and spinal cord, while GalR2 has a wider distribution in the brain, pituitary gland, and peripheral tissues (Howard et al. (1997) FEBS Lett 405: 285-90; Wang et al. (1997) J. Biol. Chem. 272: 31949-52; Depczynski et al. (1998) Ann. N Y. Acad. Sci. 863: 120-8; Habert-Ortoli et al. (1994) Proc. Natl. Acad. Sci. USA 91: 9780-3; Fathi et al. (1997) Brain Res. Mol. Brain Res. 51: 49-59). GalR3 is expressed at moderate levels only in discrete regions of the brain, and at very low levels in many central and peripheral tissues (Fathi et al. (1997) Brain Res. Mol. Brain Res. 51: 49-59; Smith et al. (1998) J. Biol. Chem. 273: 23321-6; Waters and Krause (2000) Neuroscience 95: 265-71). GalR1 was recently reported to be expressed in cholangiocytes, where Gal is increased in experimental cholestasis, mediating cholangiocyte proliferation (McMillin et al. (2017) Am. J. Pathol. 187: 819-830). All known Gal receptors are 7-transmembrane GPCRs with very different G-protein coupling and signaling functions, contributing to the diversity of Gal-mediated effects (Lang et al. (2015) Pharmacol. Rev. 67: 118-75). GalR1 signals through G_{i/o} protein, followed by cAMP and CREB pathway, while GalR2 can activate G_{12/13} and G_{q/11} in addition to (Lang et al. (2015) Pharmacol. Rev. 67: 118-75).

In the liver, a significant amount of Gal is produced and released into the systemic circulation during sympathetic nerve stimulation (Kowalyk et al. (1992) Am. J. Physiol. 262: E671-8). Endogenous hepatic Gal acts directly on the liver to selectively modulate norepinephrine's metabolic action (Mundinger and Taborsky, Jr. (2000) Am. J. Physiol. Endocrinot Metab. 278: E390-7). It was recently demonstrated that Gal stimulates cholangiocyte proliferation via GalR1-mediated ERK1/2-RKS-CREB signaling pathway in a rodent model of cholestasis (McMillin et al. (2017) Am. J. Pathol. 187: 819-830). However, the role of Gal signaling in fibrogenesis is not known. Further, the role of Gal and its' receptors in hepatic stellate cells (HSC) or hepatocytes is poorly understood. Only once the role of Gal and its receptors GalR1 and GalR2 in cholangiocyte proliferation and liver fibrosis is better understood can treatment methods tailored to this pathway be developed. Thus, there remains a need for compounds and compositions for the treatment of fibrotic disorders such as hepatic fibrosis, and methods of making and using same. These needs and other needs are satisfied by the present invention.

### SUMMARY

In accordance with the purpose(s) of the invention, as embodied and broadly described herein, the invention, in one aspect, relates to compounds and compositions for the treatment of fibrotic disorders such as, for example, hepatic fibrosis.

Thus, disclosed are methods for treating liver fibrosis in a subject, the method comprising administering to the subject an effective amount of at least one agent that modulates galanin receptor 1 (GalR1) and galanin receptor 2 (GalR2), or a pharmaceutically acceptable salt thereof.

Also disclosed are methods for treating a fibrotic disorder in a subject, the method comprising co-administering to the subject an effective amount of at least one agent that modulates GalR1, or a pharmaceutically acceptable salt thereof, and at least one agent that modulates GalR2, or a pharmaceutically acceptable salt thereof.

Also disclosed are pharmaceutical compositions comprising: (a) at least one agent that modulates GalR1, or a pharmaceutically acceptable salt thereof; (b) at least one agent that modulates GalR2, or a pharmaceutically acceptable salt thereof; and (c) a pharmaceutically acceptable carrier, wherein at least one of the agent that modulates GalR1 and the agent that modulates GalR2 is present in an effective amount.

Also disclosed are methods for making a pharmaceutical composition, the method comprising combining: (a) at least one agent that modulates Galanin receptor 1 (GalR1), or a pharmaceutically acceptable salt thereof; (b) at least one agent that modulates Galanin receptor 2 (GalR2), or a pharmaceutically acceptable salt thereof; and (c) a pharmaceutically acceptable carrier, wherein at least one of the agent that modulates GalR1 and the agent that modulates GalR2 is present in an effective amount.

Also disclosed are kits comprising: (a) at least one agent that modulates GalR1 and GalR2, or a pharmaceutically acceptable salt thereof; or (b) at least one agent that modulates GalR1, or a pharmaceutically acceptable salt thereof, and at least one agent that modulates GalR2, or a pharmaceutically acceptable salt thereof, and one or more of: (c) at least one agent known to treat a fibrotic disorder; and (d) instructions for treating a fibrotic disorder.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1A-F show representative data demonstrating that galanin expression in mouse liver during lifetime correlates with bile duct formation, being increased in male and female Mdr2KO as compared to FVBN control mice.

FIG. 2A-C shows representative low magnification pictures of galanin colocalization with markers of cholangiocytes (CK19), hepatic stellate cells (desmin), and hepatocytes (CK8) in liver tissue from male and female, FVBN and Mdr2KO mice, at 2 months old.

FIG. 3A-C show representative data demonstrating the distribution of galanin (Gal) and its receptors GalR1 and GalR2 in hepatic cells by laser capture microdissection (LCM).

FIG. 4 shows representative images illustrating the distribution of galanin in hepatic cells as determined by immunofluorescence.

FIG. 5A-F show representative data demonstrating that galanin treatment increases intrahepatic bile duct mass (IBDM) and cholangiocyte proliferation in male and female Mdr2KO and FVBN mice.

FIG. 6A-D show representative data demonstrating expression of hepatic fibrosis genes at mRNA level in liver tissue from male and female, FVBN and Mdr2KO mice, 2 mo old, treated with galanin.

FIG. 7A-F show representative data demonstrating that galanin stimulates liver fibrosis. Desmin, aSMA IHC and Sirius Red staining in liver of male and female Mdr2KO and control mice treated with vehicle or galanin.

FIG. 8A-F show representative data demonstrating that galanin receptor 1 (GalR1) vivo morpholino reduces markers of biliary hyperplasia and fibrosis in liver of control and cholestatic mice.

FIG. 9A-H show representative data demonstrating that GalR1 knockdown in Mdr2KO mice with GalR1 vivo morpholino results in reduced IBDM and slight decrease in liver fibrosis.

FIG. 10A-F show representative data demonstrating aSMA and desmin IHC in FVBN and Mdr2KO mice treated with M871.

FIG. 11A-H show representative data demonstrating galanin receptor 2 (GalR2) antagonist (M871) has no effect on biliary hyperplasia but alleviates liver fibrosis in Mdr2KO mice.

FIG. 12A-F show representative data demonstrating the effect of M40 antagonist of GalR1 and GalR2 reduces markers of biliary hyperplasia and hepatic fibrosis in FVBN and Mdr2KO mice.

FIG. 13A-H show representative data demonstrating that GalR1 and GalR2 nonspecific antagonist (M40) reduces biliary hyperplasia and liver fibrosis in Mdr2KO mice.

FIG. 14 shows representative data demonstrating that GalR1 and GalR2 antagonists alleviate serum level of Gal in Mdr2KO mice.

FIG. 15A-C show representative data demonstrating that galanin increases intrahepatic bile duct mass (IBDM) and liver fibrosis in Mdr2KO mice.

FIG. 16A and FIG. 16B show representative data demonstrating the effect of galanin on expression of genes associated with hepatic fibrosis in FVBN and Mdr2KO mice.

FIG. 17A-C show representative data demonstrating that GalR1 is essential for cholangiocytes to mediate LX-2 cell activation by an autocrine and paracrine process.

FIG. 18A-F show representative data demonstrating that GalR2 is essential for LX-2 cells to become activated by galanin directly or via cholangiocyte-conditioned media.

FIG. 19A and FIG. 19B show representative data demonstrating the effect of M40 antagonist of GalR1 and GalR2 on activation of LX-2 cells, when applied to LX-2 cells directly or via cholangiocyte-conditioned media.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Unless defined otherwise, all technical and scientific terms used herein have the same meaning as commonly understood by one of ordinary skill in the art to which this invention belongs. In this specification and in the claims which follow, reference will be made to a number of terms which shall be defined herein.

### A. Definitions

As used in the specification and the appended claims, the singular forms “a,” “an,” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X, and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “an agent that modulates GalR1 and GalR2” means an agent that is non-selective for GalR1 over GalR2 (i.e., a non-selective modulator). Examples of agents that modulate GalR1 and GalR2 include, but are not limited to, M40.

As used herein, the term “an agent that modulates GalR1” means an agent that selectively modulates GalR1 in preference over other galanin receptor subtypes, e.g., GalR2. Thus, in various aspects, an agent that modulates GalR1 may have at least about 20%, at least about 30%, at least about 40%, at least about 50%, at least about 60%, at least about 70%, at least about 80%, at least about 90%, at least about 95%, at least about 99%, or greater than about 99% selectivity in preference for GalR1 over GalR2. Examples, of agents that modulate GalR1 include, but are not limited to, a vivo-morpholino sequence and a GalR1-specific siRNA.

As used herein, the term “an agent that modulates GalR2” means an agent that selectively modulates GalR2 in preference over other galanin receptor subtypes, e.g., GalR1. Thus, in various aspects, an agent that modulates GalR2 may have at least about 20%, at least about 30%, at least about 40%, at least about 50%, at least about 60%, at least about 70%, at least about 80%, at least about 90%, at least about 95%, at least about 99%, or greater than about 99% selectivity in preference for GalR2 over GalR1. Examples, of agents that modulate GalR2 include, but are not limited to, M871.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “individually effective amount” refers to an amount of a single component, e.g., an agent that modulates GalR1, in isolation, that is sufficient to achieve the desired result or to have an effect on an undesired condition. For example, an “individually therapeutically effective amount” refers to an amount of a single component that is sufficient to achieve the desired therapeutic result or to have an effect on undesired symptoms, but is generally insufficient to cause adverse side effects.

As used herein, the term “combinatorically effective amount” refers to an amount of multiple components, e.g., an agent that modulates GalR1 and an agent that modulates GalR2, together, that is sufficient to achieve the desired result or to have an effect on an undesired condition. For example, a “combinatorically therapeutically effective amount” refers to an amount of multiple components in total that is sufficient to achieve the desired therapeutic result or to have an effect on undesired symptoms, but is generally insufficient to cause adverse side effects.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Pharmaceutical Compositions

In one aspect, disclosed are pharmaceutical compositions comprising: (a) at least one agent that modulates GalR1, or a pharmaceutically acceptable salt thereof; (b) at least one agent that modulates GalR2, or a pharmaceutically acceptable salt thereof; and (c) a pharmaceutically acceptable carrier, wherein at least one of the agent that modulates GalR1 and the agent that modulates GalR2 is present in an effective amount.

In one aspect, disclosed are pharmaceutical compositions comprising an effective amount of at least one agent that modulates GalR1 and GalR2, or a pharmaceutically acceptable salt thereof, and a pharmaceutically acceptable carrier.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa. 1990.

In a further aspect, the pharmaceutical composition is used to treat a fibrotic disorder. Examples of fibrotic disorders include, but are not limited to, hypertrophic scar, systemic sclerosis, pulmonary arterial hypertension, cardiac fibrosis, hypertrophic cardiomyopathy, cardiac dysfunction, valvular disease, arrhythmia, myelofibrosis, myelodysplastic syndrome, chronic myelogenous leukemia, cirrhosis, portal hypertension, hepatocellular carcinoma, retroperitoneal fibrosis, intestinal fibrosis, enteropathies, inflammatory bowel disease, arthrofibrosis, glial scar, Alzheimer's disease, subretinal fibrosis, epiretinal fibrosis, idiopathic pulmonary fibrosis, cystic fibrosis, pulmonary hypertension, thromboembolic disease, emphysema, mediastinal fibrosis, pancreatic fibrosis, chronic pancreatitis, duct obstruction, renal fibrosis, nephrogenic systemic fibrosis, chronic kidney disease, renal anemia, and liver fibrosis. In a still further aspect, the fibrotic disorder is liver fibrosis.

In a further aspect, the fibrotic disorder is found in the liver, the lung, the cardiac muscle, the kidney, the skin, or the eye. In a still further aspect, the fibrotic disorder is found in the liver.

In a further aspect, both the agent that modulates GalR1 and the agent that modulates GalR2 are present in a combinatorically effective amount. In a still further aspect, both the agent that modulates GalR1 and the agent that modulates GalR2 are present in individually effective amounts. In yet a further aspect, the effective amount is a therapeutically effective amount. In an even further aspect, the effective amount is a prophylactically effective amount.

1. Agents that Modulate GalR1 and GalR2

In various aspects, the invention relates to pharmaceutical compositions comprising at least one agent that modulates GalR1 and GalR2, or a pharmaceutically acceptable salt thereof. In various further aspect, the agent that modulates GalR1 and GalR2 decreases or inhibits the expression of both GalR1 and GalR2. Thus, in various further aspects, the agent that modulates GalR1 and GalR2 is an antagonist of GalR1 and GalR2. In a still further aspect, the agent that modulates GalR1 and GalR2 is M40. In yet a further aspect, the agent that modulates GalR1 and GalR2 has a sequence of

(SEQ ID NO: 1)
GWTLNSAGYLLGPPPALALA.

An agent that decreases or inhibits the expression or activity of GalR1 and GalR2 is an agent that measurably decreases or reduces the amount of mRNA encoding GalR1 and the amount of mRNA encoding GalR2, the amount of GalR1 protein and the amount of GalR2 protein, or the activity of GalR1 and the activity of GalR2 as compared to a cell not contacted with the inhibitory agent. In various aspects, the inhibitory agent results in at least a 2-fold, 3-fold, 4-fold, 5-fold, or 10-fold decrease in both GalR1 and GalR2 expression or activity.

In addition to inhibiting expression, the present invention also includes agents that inhibit the activity of GalR1 and GalR2. Inhibition of GalR1 and GalR2 activity includes inhibition of protein activity and interruption of protein interaction with other proteins, e.g., using a peptide or small molecule compound that binds specifically to both a GalR1 binding or active domain and a GalR2 binding or active domain.

In a further aspect, the agent that modulates GalR1 and GalR2 is present in an effective amount. In a still further aspect, the effective amount is a therapeutically effective amount. In yet a further aspect, the effective amount is a prophylactically effective amount.

2. Agents that Modulate GalR1

In various aspects, the invention relates to pharmaceutical compositions comprising at least one agent that modulates GalR1, or a pharmaceutically acceptable salt thereof. In various further aspect, the agent that modulates GalR1 decreases or inhibits the expression of GalR1. Thus, in various further aspects, the agent that modulates GalR1 is a GalR1 antagonist. In a still further aspect, the GalR1 antagonist is a vivo-morpholino sequence or a GalR1-specific siRNA. Examples of vivo-morpholino sequences include, but are not limited to, TTCACCATAGCCAGTTCCATCACTT (SEQ ID NO:2) and AGTTGTGCCAGCCAGGGAAAACT (SEQ ID NO:3). Examples of siRNAs include, but are not limited to,

(SEQ ID NO: 4)
CCGGCAAGTGTTCAAGTGTCACATTCTCGAGAATGTGACACTTGAACACT
TGTTTTT.

An agent that decreases or inhibits the expression or activity of GalR1 is an agent that measurably decreases or reduces the amount of mRNA encoding GalR1, the amount of GalR1 protein, or the activity of GalR1 as compared to a cell not contacted with the inhibitory agent. In various aspects, the inhibitory agent results in at least a 2-fold, 3-fold, 4-fold, 5-fold, or 10-fold decrease in GalR1 expression or activity.

In addition to inhibiting expression, the present invention also includes agents that inhibit the activity of GalR1. Inhibition of GalR1 activity includes inhibition of protein activity and interruption of protein interaction with other proteins, e.g., using a peptide or small molecule compound that binds specifically to a GalR1 binding or active domain.

In a further aspect, the agent that modulates GalR1 is present in an effective amount. In a still further aspect, the effective amount is a therapeutically effective amount. In yet a further aspect, the effective amount is a prophylactically effective amount. In an even further aspect, the effective amount is an individually effective amount.

3. Agents that Modulate GalR2

In various aspects, the invention relates to pharmaceutical compositions comprising at least one agent that modulates GalR2, or a pharmaceutically acceptable salt thereof. In various further aspect, the agent that modulates GalR2 decreases or inhibits the expression of GalR2. Thus, in various further aspects, the agent that modulates GalR2 is a GalR2 antagonist. In a still further aspect, the GalR2 antagonist is M871. In yet a further aspect, the agent that modulates GalR2 has a sequence of

(SEQ ID NO: 5)
WTLNSAGYLLGPEHPPPALALA.

An agent that decreases or inhibits the expression or activity of GalR2 is an agent that measurably decreases or reduces the amount of mRNA encoding GalR2, the amount of GalR2 protein, or the activity of GalR2 as compared to a cell not contacted with the inhibitory agent. In various aspects, the inhibitory agent results in at least a 2-fold, 3-fold, 4-fold, 5-fold, or 10-fold decrease in GalR2 expression or activity.

In addition to inhibiting expression, the present invention also includes agents that inhibit the activity of GalR2. Inhibition of GalR2 activity includes inhibition of protein activity and interruption of protein interaction with other proteins, e.g., using a peptide or small molecule compound that binds specifically to a GalR2 binding or active domain.

In a further aspect, the agent that modulates GalR2 is present in an effective amount. In a still further aspect, the effective amount is a therapeutically effective amount. In yet a further aspect, the effective amount is a prophylactically effective amount. In an even further aspect, the effective amount is an individually effective amount.

### C. Methods of Preparing a Composition

In one aspect, disclosed are methods for making a pharmaceutical composition, the method comprising combining: (a) at least one agent that modulates GalR1, or a pharmaceutically acceptable salt thereof (b) at least one agent that modulates GalR2, or a pharmaceutically acceptable salt thereof; and (c) a pharmaceutically acceptable carrier, wherein at least one of the agent that modulates GalR1 and the agent that modulates GalR2 is present in an effective amount.

In a further aspect, the effective amount is a prophylactically effective amount. In a still further aspect, the effective amount is a therapeutically effective amount.

In a further aspect, combining is co-formulation of the agent that modulates GalR1 and the agent that modulates GalR2 with the pharmaceutically acceptable carrier. In a still further aspect, the solid dosage form is a tablet. In yet a further aspect, the solid dosage form is a capsule.

In a further aspect, co-formulation provides an oral dosage form comprising the agent that modulates GalR1, the agent that modulates GalR2, and the pharmaceutically acceptable carrier. In a still further aspect, the oral dosage form is an oral solid dosage form.

In a further aspect, co-formulation provides an injectable dosage form comprising the agent that modulates GalR1, the agent that modulates GalR2, and the pharmaceutically acceptable carrier.

### D. Methods of Using the Compositions

Also provided are methods of use of a disclosed composition or medicament. In one aspect, the method of use is directed to the treatment of a disorder. In a further aspect, the disclosed compounds can be used as single agents or in combination with one or more other drugs in the treatment, prevention, control, amelioration, or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

1. Treatment Methods

In one aspect, the compounds and compositions disclosed herein are useful for treating, preventing, ameliorating, controlling or reducing the risk of a variety of fibrotic disorders, including, but not limited to, hypertrophic scar, systemic sclerosis, pulmonary arterial hypertension, cardiac fibrosis, hypertrophic cardiomyopathy, cardiac dysfunction, valvular disease, arrhythmia, myelofibrosis, myelodysplastic syndrome, chronic myelogenous leukemia, cirrhosis, portal hypertension, hepatocellular carcinoma, retroperitoneal fibrosis, intestinal fibrosis, enteropathies, inflammatory bowel disease, arthrofibrosis, glial scar, Alzheimer's disease, subretinal fibrosis, epiretinal fibrosis, idiopathic pulmonary fibrosis, cystic fibrosis, pulmonary hypertension, thromboembolic disease, emphysema, mediastinal fibrosis, pancreatic fibrosis, chronic pancreatitis, duct obstruction, renal fibrosis, nephrogenic systemic fibrosis, chronic kidney disease, renal anemia, and liver fibrosis.

The compounds and compositions are further useful in methods for the prevention, treatment, control, amelioration, or reduction of risk of fibrotic disorders noted herein. The compounds and compositions are further useful in a method for the prevention, treatment, control, amelioration, or reduction of risk of the aforementioned fibrotic disorders in combination with other agents.

In one aspect, the disclosed compounds can be used in combination with one or more other drugs in the treatment, prevention, control, amelioration, or reduction of risk of fibrotic disorders for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions include those that contain one or more other active ingredients, in addition to a compound of the present invention.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of fibrotic disorders for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be co-administered, either in concomitant therapy or in a fixed combination.

a. Treating Liver Fibrosis

In one aspect, disclosed are methods for treating liver fibrosis in a subject, the method comprising administering to the subject an effective amount of at least one agent that modulates galanin receptor 1 (GalR1) and galanin receptor 2 (GalR2), or a pharmaceutically acceptable salt thereof.

In a further aspect, modulates is inhibits. In a still further aspect, modulates is decreases the activity of.

In a further aspect, the subject is a mammal. In a still further aspect, the subject is human.

In a further aspect, the subject has been diagnosed with a need for treatment of liver fibrosis prior to the administering step. In a still further aspect, the subject is at risk for developing liver fibrosis prior to the administering step.

In a further aspect, the method further comprises identifying a subject in need of treatment of liver fibrosis.

In a further aspect, the agent that modulates GalR1 and GalR2 is an antagonist of GalR1 and GalR2. In a still further aspect, the agent that modulates GalR1 and GalR2 is M40.

In a further aspect, the effective amount is a therapeutically effective amount. In a still further aspect, the effective amount is a prophylactically effective amount.

b. Treating a Fibrotic Disorder

In one aspect, disclosed are methods for treating a fibrotic disorder in a subject, the method comprising co-administering to the subject an effective amount of at least one agent that modulates GalR1, or a pharmaceutically acceptable salt thereof, and at least one agent that modulates GalR2, or a pharmaceutically acceptable salt thereof. Examples of fibrotic disorders include, but are not limited to, hypertrophic scar, systemic sclerosis, pulmonary arterial hypertension, cardiac fibrosis, hypertrophic cardiomyopathy, cardiac dysfunction, valvular disease, arrhythmia, myelofibrosis, myelodysplastic syndrome, chronic myelogenous leukemia, cirrhosis, portal hypertension, hepatocellular carcinoma, retroperitoneal fibrosis, intestinal fibrosis, enteropathies, inflammatory bowel disease, arthrofibrosis, glial scar, Alzheimer's disease, subretinal fibrosis, epiretinal fibrosis, idiopathic pulmonary fibrosis, cystic fibrosis, pulmonary hypertension, thromboembolic disease, emphysema, mediastinal fibrosis, pancreatic fibrosis, chronic pancreatitis, duct obstruction, renal fibrosis, nephrogenic systemic fibrosis, chronic kidney disease, renal anemia, and liver fibrosis. In a further aspect, the fibrotic disorder is liver fibrosis.

In a further aspect, modulates is inhibits. In a still further aspect, modulates is decreases the activity of.

In a further aspect, the agent that modulates GalR1 is a GalR1 antagonist. In a still further aspect, the GalR1 antagonist is a vivo-morpholino sequence or a GalR1-specific siRNA.

In a further aspect, the agent that modulates GalR2 is a GalR2 antagonist. In a still further aspect, the GalR2 antagonist is M871.

In a further aspect, the at least one agent that modulates GalR1 is a GalR1 antagonist and wherein the at least one agent that modulates GalR2 is a GalR2 antagonist.

In a further aspect, the fibrotic disorder is found in the liver, the lung, the cardiac muscle, the kidney, the skin, or the eye. In a still further aspect, the fibrotic disorder is found in the liver.

In a further aspect, the subject is a mammal. In a still further aspect, the subject is human.

In a further aspect, the subject has been diagnosed with a need for treatment of a fibrotic disorder prior to the administering step. In a still further aspect, the subject is at risk for developing a fibrotic disorder prior to the administering step.

In a further aspect, the agent that modulates GalR1 and the agent that modulates GalR2 are co-formulated. In a still further aspect, the agent that modulates GalR1 and the agent that modulates GalR2 are co-packaged.

In a further aspect, the method further comprises identifying a subject in need of treatment of a fibrotic disorder.

In a further aspect, the effective amount is a therapeutically effective amount. In a still further aspect, the effective amount is a prophylactically effective amount.

In a further aspect, the effective amount is an individually effective amount of the agent that modulates GalR1 or the agent that modulates GalR2. In a still further aspect, the effective amount is an individually effective amount of the agent that modulates GalR1. In yet a further aspect, the effective amount is an individually effective amount of the agent that modulates GalR2.

In a further aspect, the effective amount is a combinatorically effective amount of the agent that modulates GalR1 and the agent that modulates GalR2.

2. Manufacture of a Medicament

In one aspect, the invention relates to a medicament comprising one or more agents that inhibit the expression of GalR1, or a pharmaceutically acceptable salt thereof; and one or more agents that inhibit the expression of GalR2, or a pharmaceutically acceptable salt thereof.

In various aspect, the invention relates methods for the manufacture of a medicament for treating a fibrotic disorder comprising combining one or more disclosed compounds, products, or compositions or a pharmaceutically acceptable salt thereof, with a pharmaceutically acceptable carrier. It is understood that the disclosed methods can be performed with the disclosed compounds, products, and pharmaceutical compositions. It is also understood that the disclosed methods can be employed in connection with the disclosed methods of using.

3. Use of Compounds and Compositions

Also provided are the uses of the disclosed compounds and compositions. Thus, in one aspect, disclosed are uses of at least one agent that modulates GalR1 and GalR2, or a pharmaceutically acceptable salt thereof. Also disclosed are uses of at least one agent that modulates GalR1, or a pharmaceutically acceptable salt thereof; and at least one agent that modulates GalR2, or a pharmaceutically acceptable salt thereof.

In a further aspect, disclosed are uses of at least one agent that modulates GalR1, or a pharmaceutically acceptable salt thereof, and at least one agent that modulates GalR2, or a pharmaceutically acceptable salt thereof, in the manufacture of a medicament for the treatment of a fibrotic disorder.

In a further aspect, the use relates to a process for preparing a pharmaceutical composition comprising a therapeutically effective amount of at least one agent that modulates GalR1, or a pharmaceutically acceptable salt thereof, and at least one agent that modulates GalR2, or a pharmaceutically acceptable salt thereof, for use as a medicament.

In a further aspect, the use relates to a process for preparing a pharmaceutical composition comprising a therapeutically effective amount of at least one agent that modulates GalR1, or a pharmaceutically acceptable salt thereof, and at least one agent that modulates GalR2, or a pharmaceutically acceptable salt thereof, wherein a pharmaceutically acceptable carrier is intimately mixed with a therapeutically effective amount of the at least one agent that modulates GalR1 or at least one agent that modulates GalR2.

In various aspects, the use relates to the treatment of a fibrotic disorder in a vertebrate animal. In a further aspect, the use relates to the treatment of a fibrotic disorder in a human subject.

In a further aspect, the use is the treatment of a fibrotic disorder. In a still further aspect, the fibrotic disorder is liver fibrosis.

It is understood that the disclosed uses can be employed in connection with the disclosed compounds, methods, compositions, and kits. In a further aspect, disclosed are uses of a disclosed compound or composition of a medicament for the treatment of a fibrotic disorder in a mammal.

In a further aspect, disclosed are uses of a disclosed compound or composition in the manufacture of a medicament for the treatment of a fibrotic disorder selected from hypertrophic scar, systemic sclerosis, pulmonary arterial hypertension, cardiac fibrosis, hypertrophic cardiomyopathy, cardiac dysfunction, valvular disease, arrhythmia, myelofibrosis, myelodysplastic syndrome, chronic myelogenous leukemia, cirrhosis, portal hypertension, hepatocellular carcinoma, retroperitoneal fibrosis, intestinal fibrosis, enteropathies, inflammatory bowel disease, arthrofibrosis, glial scar, Alzheimer's disease, subretinal fibrosis, epiretinal fibrosis, idiopathic pulmonary fibrosis, cystic fibrosis, pulmonary hypertension, thromboembolic disease, emphysema, mediastinal fibrosis, pancreatic fibrosis, chronic pancreatitis, duct obstruction, renal fibrosis, nephrogenic systemic fibrosis, chronic kidney disease, renal anemia, and liver fibrosis. In a still further aspect, the fibrotic disorder is liver fibrosis.

In a further aspect, the fibrotic disorder is found in the liver, the lung, the cardiac muscle, the kidney, the skin, or the eye. In a still further aspect, the fibrotic disorder is found in the liver.

In a further aspect, disclosed are uses of a disclosed compound or composition in the manufacture of a medicament for the treatment of a fibrotic disorder.

In various aspects, the agents and methods described herein can be used prophylactically, such as to prevent, reduce or delay progression of a fibrotic disorder.

4. Kits

In one aspect, disclosed are kits comprising: (a) at least one agent that modulates GalR1 and GalR2, or a pharmaceutically acceptable salt thereof; or (b) at least one agent that modulates GalR1, or a pharmaceutically acceptable salt thereof, and at least one agent that modulates GalR2, or a pharmaceutically acceptable salt thereof, and one or more of: (c) at least one agent known to treat a fibrotic disorder; and (d) instructions for treating a fibrotic disorder.

In various aspects, the agents and pharmaceutical compositions described herein can be provided in a kit. The kit can also include combinations of the agents and pharmaceutical compositions described herein. The kit can include: a) one or more agents, such as in a composition that includes the agents; b) informational material; and any combination of a) and b). The informational material can be descriptive, instructional, marketing or other material that relates to the methods described herein and/or to the use of the agents for the methods described herein. For example, the informational material relates to the use of the agents herein to treat a subject who has, or who is at risk for developing, a fibrotic disorder.

In various aspects, the informational material can include instructions for administering the pharmaceutical composition and/or cell(s) in a suitable manner to treat a human, e.g., in a suitable dose, dosage form, or mode of administration (e.g., a dose, dosage form, or mode of administration described herein). In a further aspect, the informational material can include instructions to administer the pharmaceutical composition to a suitable subject, e.g., a human having, or at risk for developing, a fibrotic disorder.

In various aspects, the composition of the kit can include other ingredients, such as a solvent or buffer, a stabilizer, a preservative, a fragrance or other cosmetic ingredient. In such aspects, the kit can include instructions for admixing the agent and the other ingredients, or for using one or more compounds together with the other ingredients.

In a further aspect, the agent that modulates GalR1 and the agent that modulates GalR2 are co-formulated. In a still further aspect, the agent that modulates GalR1 and the agent that modulates GalR2 are co-packaged.

In a further aspect, the agent known to treat a fibrotic disorder is selected from a TGF-β inhibitor (SHP-627, hydronidone, PXS-25, disitertide, fresolimumab, LY2382770), an integrin αvβ6 inhibitor (e.g., STX-100, CWHM-12), an ALK5 antagonist (e.g., SB-431542), a BMP-7 agonist (e.g., THR-184), a CTGF inhibitor (e.g., PF-06473871, RXI-109, FG-3019), a PDGFR antagonist (e.g., imatinib, BOT-191, nilotinib, dasatinib), a VEGFR/PDGFR antagonist (e.g., nintedanib, sorafenib), a TNF inhibitor (e.g., thalidomide, pomalidomide, etanercept, belimumab), a HGF stimulant (e.g., refanalin), an interleukin inhibitor (e.g., dectrekumab, tralokinumab, SAR156597), an interleukin antagonist (e.g., anakinra, rilonacept), a CC chemokine inhibitor (e.g., carlumab, bindarit), a CC chemokine antagonist (maraviroc, RS-504393), an interferon stimulant (e.g., actimmune, interferon alpha oral lozenge), a MMP/TIMP inhibitor (e.g., batimastat, marimastat), an endothelin antagonist (e.g., macitentan, bosentan, ambrisentan, sparsentan, atrasentan), an angiotensin II antagonist (e.g., losartan), a GPCR antagonist (e.g., BMS-986020, SAR-100842, PAR1 antagonism, curcumin, silymarin), a GPCR agonist (e.g., β-caryophyllene, beraprost, iloprost, treprostinil, aviptadil), a leukocyte elastase inhibitor (e.g., sivelestat), a TAFI inhibitor (e.g., UK-396082), and a relaxin stimulant (e.g., serelaxin).

In a further aspect, the agent known to treat a fibrotic disorder is an anti-inflammatory. Examples of anti-inflammatories include, but are not limited to, ibuprofen, aspirin, naproxen sodium, oxaprozin, etodolac, indomethacin, naproxen, nabumetone, diclofenac, and vimovo.

In a further aspect, the kit further comprises a plurality of dosage forms, the plurality comprising one or more doses; wherein each dose comprises the agent that modulates GalR1 and GalR2, and the agent known to treat a fibrotic disorder, wherein at least one is present in an effective amount. In a still further aspect, the effective amount is a therapeutically effective amount. In yet a further aspect, the effective amount is a prophylactically effective amount. In an even further aspect, each dose of the agent that modulates GalR1 and the agent that modulates GalR2 are co-packaged. In a still further aspect, each dose of the agent that modulates GalR1 and the agent that modulates GalR2 are co-formulated.

In a further aspect, the kit further comprises a plurality of dosage forms, the plurality comprising one or more doses; wherein each dose comprises the agent that modulates GalR1, the agent that modulates GalR2, and the at least one agent known to treat a fibrotic disorder; wherein at least one is present in an effective amount. In a still further aspect, the effective amount is a therapeutically effective amount. In yet a further aspect, the effective amount is a prophylactically effective amount. In an even further aspect, each dose of the agent that modulates GalR1, the agent that modulates GalR2, and the agent known to treat a fibrotic disorder are co-packaged. In a still further aspect, each dose of the agent that modulates GalR1, the agent that modulates GalR2, and the agent known to treat a fibrotic disorder are co-formulated.

In a further aspect, the dosage forms are formulated for oral administration. In a still further aspect, the dosage forms are formulated for intravenous administration.

### 5. Subjects

In various aspects, the subject of the herein disclosed methods is a vertebrate, e.g., a mammal. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with a fibrotic disorder prior to the administering step. In some aspects of the disclosed methods, the subject has been identified with a need for treatment prior to the administering step. In one aspect, a subject can be treated prophylactically with a compound or composition disclosed herein, as discussed herein elsewhere.

a. Dosage

Toxicity and therapeutic efficacy of the agents and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, Pa.). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, N.Y.).

b. Routes of Administration

Also provided are routes of administering the disclosed compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In various aspects, the modes of administration described above may be combined in any order. In this respect, one or more agents that inhibit expression or activity of GalR1 can be administered before, after, or simultaneously with one or more agents that inhibit the expression or activity of GalR2.

### E. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Materials and Methods

a. Chemicals, Kits, Antibodies, and Tissue Culture Media

All chemicals were purchased from Millipore-Sigma (Burlington, Mass.) unless otherwise stated, and were of the highest grade available. Galanin Enzyme Immunoassay (EIA) kit was purchased from Peninsula Laboratories International (San Carlos, Calif.). RNeasy kit for isolation of RNA from cells and tissue was from Qiagen, SA Biosciences (Frederik, Md.). Galanin receptor 1 (GalR1) vivo-morpholino sequences for mouse (GalR1-sequence 1: TTCACCATAGCCAGTTCCATCACTT, SEQ ID NO:2, and sequence 2: AGTTGTGCCAGCCAGGGAAAACT, SEQ ID NO:3) and a mismatch sequence (MM: TTGAGCATACCCACTTCGATCCTT, SEQ ID NO:6) were from Gene Tools (Philomath, Oreg.). GalR1 siRNA, recombinant Galanin (1-29), M617, AR-M, M871, M40 were purchased from Tocris (Minneapolis, Minn.). Hematoxylin and VectaStain kits for immunohistochemistry (IHC) staining were from Vector Laboratories (Burlingame, Calif.). In IHC and immunofluorescence (IF) assays the following antibodies were used: Gal, cytokeratin (CK)-19, CK8, desmin, alpha-smooth muscle actin (aSMA) antibodies, from Abcam (Cambridge, Mass.). Culture media including DMEM, MEM and the supplements, i.e. fetal bovine serum (FBS) and penicillin/streptomycin (P/S) were from Gibco BRL purchased through ThermoFisher Scientific (Waltham, Mass.).

b. Animal Experiments

FVB/N (FVBN) and Mdr2^{-/-} (Mdr2KO) mice were purchased from Jackson Laboratory (Bar Harbor, Me.) and maintained in a temperature-controlled environment at 20-22° C. with a 12:12 hours light-dark cycle, having free access to food and drinking water. All animal procedures were performed in accord with the guidelines of Baylor Scott and White (Temple, Tex.) Institutional Animal Care and Use Committee, with approved protocols. In time-course experiments, 1 week, 1 months, 2 months and 4 months old male and female FVBN and Mdr2KO mice were used. In experiments designed to measure the effect of various agonists and antagonists of Gal receptors, on the extent of liver fibrosis in Mdr2KO mice, 2 months old, male and female Mdr2KO mice were used, as well as FVBN mice as negative controls. Four to five animals in each group of age/gender/type (FVBN or Mdr2KO) were used. In parallel, an equal number of 2 months old, male and female FVBN and MDR2KO mice were treated with vehicle only (saline or 20% DMSO) via the same type of minipumps. Galanin, M871 and M40 peptides were dissolved in saline or 20% DMSO in saline, depending on their solubility, and administered by using Alzet osmotic minipumps (Cupertino, Calif.). Galanin, M871 and M40, were administered at a rate of 4 nmol/Kg/day (mice weights being 25-30 g). Vivo-morpholino oligos including two sequences specific to mouse GalR1, and one mouse GalR1 mismatch control, were administered at a rate of 25 μg/mouse/day, for 14 days, after which the mice were euthanized with euthasol, followed by exsanguination and collection of liver tissue.

### C. Assessment of mRNA Expression for Gal, Ck19, Asma, Collagen Type 1A1 (Col1A1), Matrix Metalloproteinase-2 (MMP-2), Tissue Inhibitor of Metalloproteinase 1 (TIMP1), GalR1 and GalR2 in Mouse Liver, or Mouse Cholangiocytes and Human Lx-2 Cells in Culture

Expression of several genes at mRNA level in liver tissue or cells in culture, was performed by real time quantitative PCR (RT-qPCR). Total RNA was isolated by using RNeasy kit, followed by cDNA synthesis with iScript kit from Bio-Rad Life Sciences (Hercules, Calif.), and RT-qPCR using iTaq Universal SYBR-Green Supermix from the same company. RTZ qPCR Primer Assays were purchased from Qiagen SA Biosciences (Frederik, Md.). A thermal cycler AriaMax Real Time PCR system from Agilent Technologies (Santa Clara, Calif.) was used for running qPCR. The data was analyzed as described (McMillin et al. (2017)Am. J. Pathol. 187: 819-830).

d. Assessment of Biliary Hyperplasia and Liver Fibrosis in MDR2KO and FVBN Mice

Biliary hyperplasia was assessed by measuring the intrahepatic biliary duct mass (IBDM) by IHC for CK19, a marker of cholangiocytes. Hepatic fibrosis markers such as aSMA and desmin were assayed by IHC of liver tissue from mice treated with GalR1 vivo-morpholino oligos, or various agonists and antagonists of Gal receptors. Thus, for IHC, liver tissue sections of 4 μm were immunolabeled with primary antibodies specific to proteins of interest, and then process for staining with VectaStain kits (Burlingame, Calif.). The IHC slides were scanned with a Leica SCN400 scanner at 20× magnification, followed by screenshots at 10× magnification, and image analysis with ImageJ software downloaded from the NIH website. For all samples and controls, the percent areas of colored pixels were calculated and compared for significant differences. Liver samples were also assayed by using Sirius Red specific staining of collagens I and III which are increased in hepatic fibrosis, with the kit from Millipore-Sigma (Burlington, Mass.).

e. Assessment of Gal Concentration in Liver Samples of MDR2KO and FVBN Mice by EIA

Galanin was assessed in liver samples of Mdr2KO and FVBN mice, by using a kit purchased from Peninsula Laboratories International (San Carlos, Calif.), according to the manufacturer's instructions.

f. Assessment of Gal, GalR1 and GalR2 Expression in Cholangiocytes, HSC and Hepatocytes in Liver Samples of MDR2KO and FVB/N Mice by Laser Capture Microdissection (LCM)

Frozen sections of liver (8 μm thick) from 2 months old male and female FVBN and Mdr2KO mice were processed for immunofluorescence (IF) by fixation with 4% paraformaldehyde, blocking of nonspecific binding with 4% bovine serum albumin (BSA) in phosphate buffer saline (PBS) supplemented with 0.5% Tween 20 (PBST), followed by incubation with 2-5 μg/mL primary antibody in PBST/BSA overnight at 4° C., and subsequent labeling with Alexa Fluor 488-conjugated secondary antibody incubation for 1 hour at room temperature. The liver sections were labeled for CK19 marker of cholangiocytes, CK8 marker of hepatocytes, and desmin of HSC. Subsequently, a Leica LMD7000 microdissection system (Temple Health & Bioscience District, Temple, Tex.) was used to isolate the specific liver cells. The RNA isolation from batches of 500-1000 cells was achieved using Arcturus PicoPure Frozen RNA Isolation kit from Thermo Fisher Scientific (Waltham, Mass.). The expression of Gal and its receptors GalR1 and GalR2 was then accomplished by the same procedure described for RT-qPCR.

g. Assessment of Gal Distribution in Different Types of Cells in Liver Tissue by Colocalization Using Confocal Microscopy

The presence of Gal inside or on the surface of cholagiocytes, HSC, and hepatocytes was evaluated by double fluorescent labeling of liver frozen sections with a mix of antibodies specific to Gal and one of the 3 cell markers, i.e., CK19 for cholangiocytes, CK8 for hepatocytes and desmin for HSC. The overlay of red fluorescence-labeled Gal with the green fluorescence-labeled cells was observed by confocal microscopy, using a laser scanning system Leica Microsystems Inc. (Buffalo Grove, Ill.).

h. Assessment of Liver Enzymes, Creatinine (Crea), Blood Urea Nitrogen (BUN) and Creatine Kinase (CK)

Serum alanine aminotransferase (ALT), aspartate aminotransferase (AST), alkaline phosphatase (ALKP), CREA, BUN and CK were measured using the IDEXX Catalyst One instrument from IDEXX Laboratories, Inc (Houston, Tex.).

i. Assessment of Cholangiocyte-Mediated Activation of HSC in Culture

In vitro experiments were run with mouse pool cholangiocytes and human LX-2 cells purchased from the American Type Culture Collection (ATCC, Manassas, Va.). The cells were grown according to the instructions from ATCC. The effect of GalR1 and GalR2 agonists and antagonists on LX-2 cell activation was tested by measuring changes in αSMA and Col1A1 expression in LX-2 cells, by RT-qPCR and immunofluorescence (IF) confocal microscopy. For IF, LX-2 cells grown on coverslips inside 6-well plates, were fixated with 4% PFA, blocked for preventing nonspecific binding, with 4% BSA in PBST, followed by incubation with 2-5 μg/mL primary antibody in PBST overnight at 4° C., and subsequent labeling with Cy3-conjugated secondary antibody incubation for 1 hour at room temperature. After washings and mounting in prolong Gold Antifade Mountant with DAPI from Thermo Fisher Sci. (Waltham, Mass.). Expression of GalR1 mRNA was downregulated in cholangiocytes by transfecting the cells with mouse GalR1-specific siRNA from OriGene (Rockville, Md.), using Lipofectamine 2000 Reagent from Thermo Fisher Sci. (Waltham, Mass.) according to manufacturer's instructions.

j. Statistics

Quantifications by RT-qPCR, EIA, and image analysis were analyzed by calculating the average and standard error of the mean (SEM) of three replicates for each group of tested animals. The number of animals (N) used for each treatment or as controls was 4-5 for each experiment. The statistical difference was calculated by using the Student's test, and was considered significant when the p value was less than 0.05. When multiple groups of animals were compared, the two-way ANOVA followed by an appropriate post-hoc test with GraphPad Prism software (San Diego, Calif.) was used.

2. Time-Course of Gal Expression in the Liver of Mdr2Ko Mice is Positively Correlated with Cholangiocyte Marker CK19 Expression

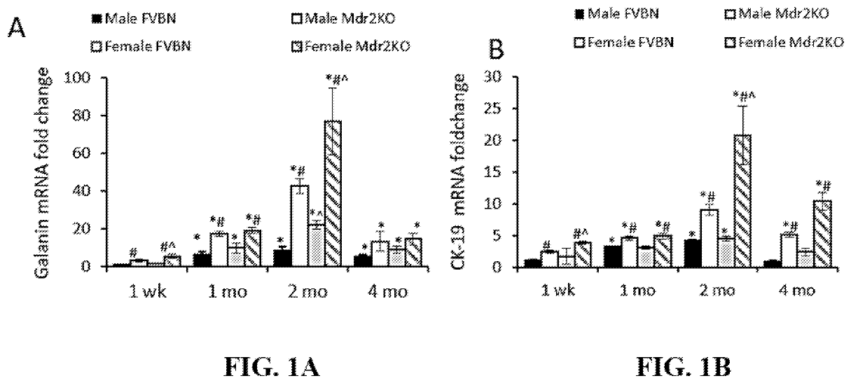

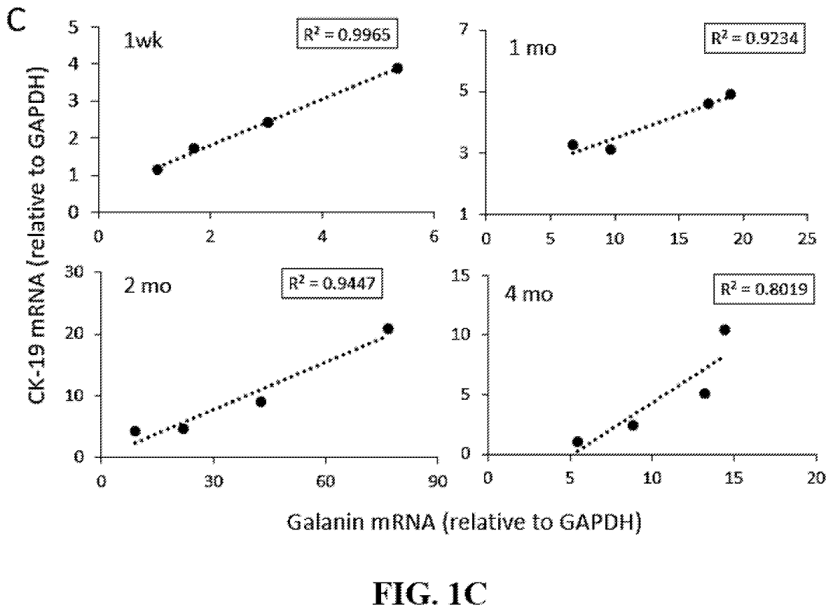

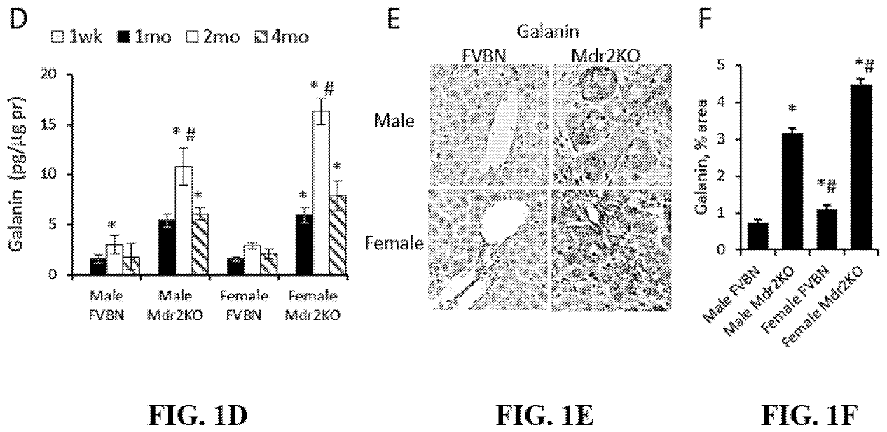

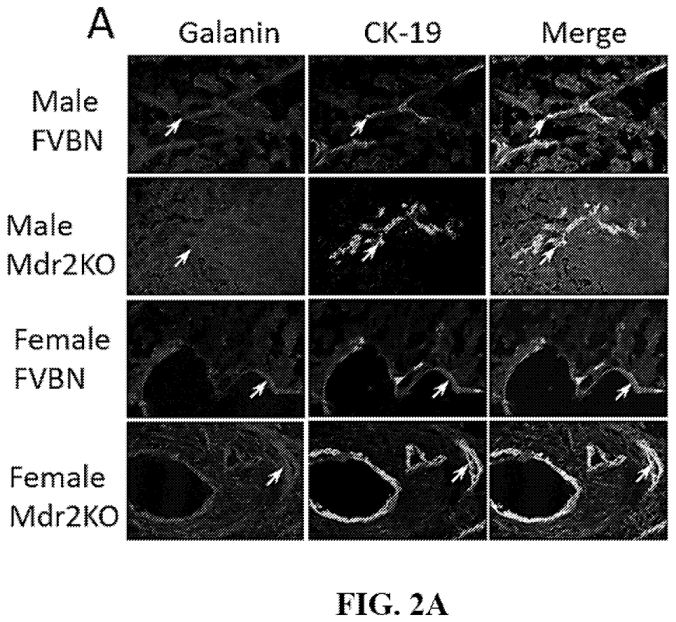

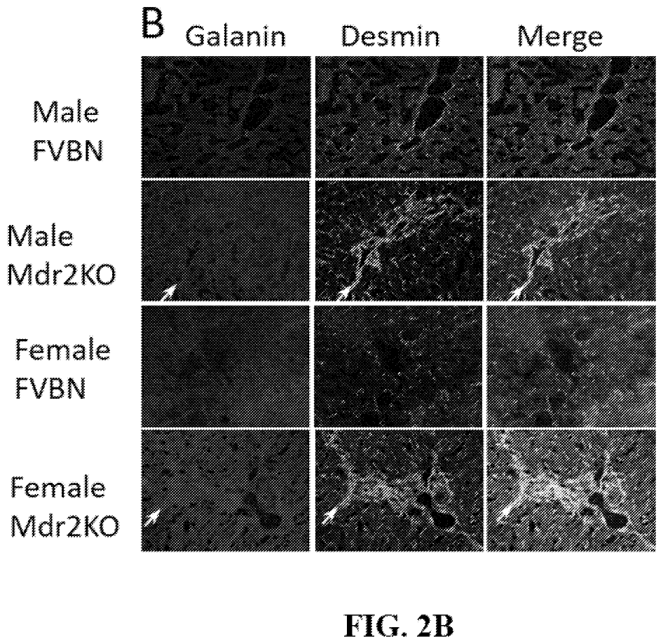

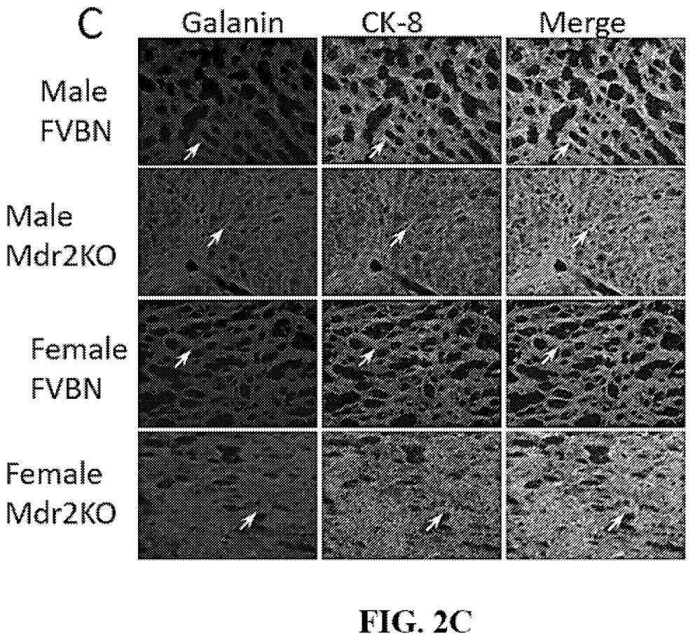

Galanin and CK19 expression at mRNA levels, were assessed in livers of male and female FVBN and Mdr2KO mice from 1 week to 4 months old. As illustrated in FIG. 1A, Gal mRNA was expressed more in Mdr2KO mice than in FVBN controls at all time points tested, and the highest levels were detected at the age of 2 months for male and female Mdr2KO mice. In both FVBN and Mdr2KO mice, Gal mRNA increased with age up to 2 months, then it regressed at 4 months. Interestingly, when, in the same liver samples, the mRNA of CK19 was assessed, a similar pattern of expression was found (FIG. 1B). There was only a slow and minimal increase of CK19 mRNA in FVBN mice vs time-course, up to 2 months, and a regression afterwards, with no gender-related differences. In Mdr2KO mice, CK19 expression was greater than in FVBN mice at every tested time-point, and it followed the same pattern as in FVBNs, rising up to 2 months then decreasing by 4 months of age. For both Gal and CK19, the mRNA expression was significantly higher in females than in males at 2 months of age. In order to quantify the correlation between galanin and CK19 in livers of male and female FVBN and Mdr2KO mice, regression correlation graphs were plotted for each age group (FIG. 1C). A positive correlation was found for wild-type and Mdr2KO mice of all ages, and the correlation coefficients were higher than 0.8019. To confirm these data, Gal peptide concentration was assayed in the liver of male and female FVBN and Mdr2KO mice at 1 week, 1 month, 2 months, and 4 months of age, by EIA (FIG. 1D). The peptide was not detected in liver from 1 week old mice, but it reached levels of 1-16 pg/μg protein in older mice, with a maximum in 2 months old Mdr2KO mice. At all tested time-points, Gal concentration in the liver was greater in Mdr2KO mice than in FVBN controls, clearly indicating a significant increase in hepatic Gal associated with cholestasis in Mdr2KO mice. Galanin assay by IHC in sections of liver from 2 months old male and female, FVBN and Mdr2KO mice, demonstrated a remarkable increase in Gal-specific staining in Mdr2KO mice as compared to FVBN mice (FIG. 1E). Image analysis of Gal IHC in these samples confirmed a significant increase in Gal in liver of Mdr2KO mice as compared to controls (FIG. 1F). These results were confirmed by IF labeling of Gal in liver sections of male and female FVBN and Mdr2KO mice (FIG. 2A-C).

Referring to FIG. 1A-F, the time-course of galanin and CK19 expression in the liver of Mdr2KO mice vs FVBN controls is shown. mRNAs of galanin (FIG. 1A) and CK19 marker of cholangiocytes (FIG. 1B) were assessed by RT-qPCR in liver of male and female, FVBN and Mdr2KO mice, from 2 weeks to 4 months old. FIG. 1C shows regression curves of CK19 mRNA vs galanin mRNA, and the correlation coefficients of FVBN and Mdr2KO mice, 1 week, 1 month, 2 months and 4 months old. FIG. 1D shows galanin concentration in liver of FVBN and Mdr2KO mice, 1 week, 1 month, 2 months, and 4 months old. FIG. 1E shows immunohistochemistry (IHC) of galanin in the liver of 2 months old male and female FVBN and Mdr2KO mice. FIG. 1F shows a bar plot of galanin measured as area percentage of stained pixels in IHC images of galanin in FIG. 1E. N=4, p<0.05.

Referring to FIG. 2A-C, double immunofluorescence labeling and confocal microscopy were used to test galanin co-localization with markers of cholangiocytes (CK19), hepatic stellate cells (desmin) and hepatocytes (CK8) in liver tissue from male and female, FVBN and Mdr2KO mice, at two months old. FIG. 2A shows IF co-localization of galanin (left panel) with CK-19 (middle panel) marker of cholangiocytes. FIG. 2B shows IF co-localization of galanin (left panel) with desmin (middle panel) marker of HSCs. FIG. 2C shows IF co-localization of galanin (left panel) with CK-8 (middle panel) marker of hepatocytes. The co-localized pixels (i.e., the pixels from both FIG. 2A and FIG. 2B) are shown in the right pane images. The arrows point to co-localized pixels.

3. Distribution of Gal and its Receptors GalR1 and GalR2 in Various Types of Hepatic Cells

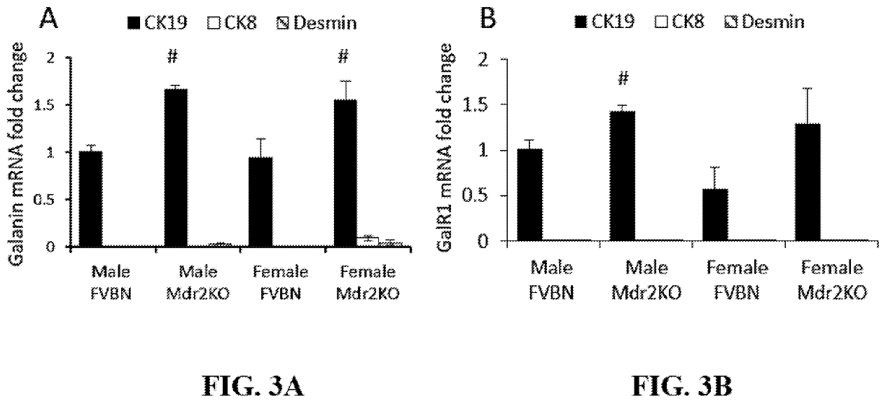

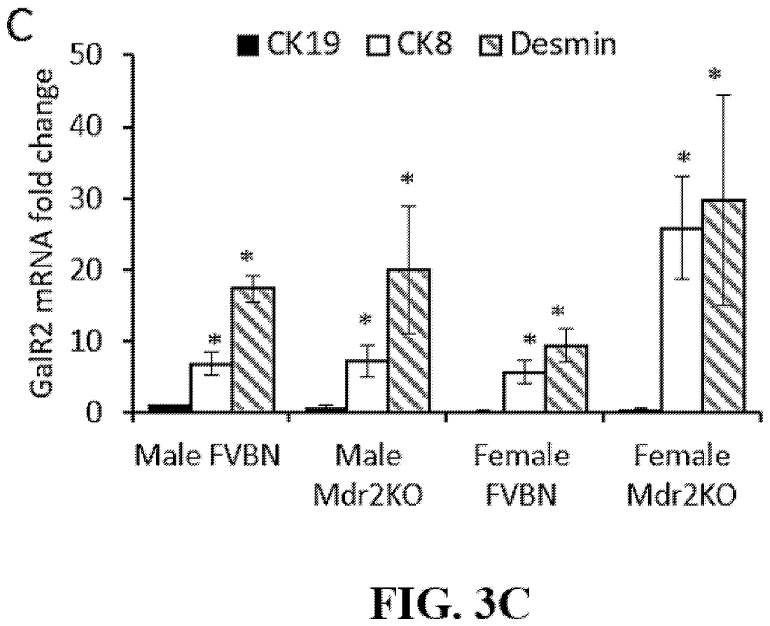

Because Gal is commonly considered to be synthesized exclusively in central or peripheral parts of the nervous system, and the notion of galanin being produced in liver cells is very new (McMillin et al. (2017)Am. J. Pathol. 187: 819-830), Gal mRNA expression was explored in cholangiocytes, HSC and hepatocytes by laser capture microdissection (LCM). As shown in FIG. 3A, Gal mRNA was detected only in cholangiocytes, in FVBN and Mdr2KO mice, 2 months old, males and females. Galanin receptors GalR1 and GalR2 were determined by the same method (FIG. 3B and FIG. 3C). The results indicate that GalR1 mRNA is expressed mostly in cholangiocytes, while GalR2 mRNA can hardly be detected in cholangiocytes, but it is expressed in hepatocytes and HSC.

Referring to FIG. 3A-C, liver frozen sections from 2 months old male and female FVBN and Mdr2KO mice were processed by LCM as described elsewhere herein. FIG. 3A shows the relative expression of galanin mRNA in CK19-immunolabeled cholangiocytes, CK8-immunolabeled hepatocytes and desmin-immunolabeled HSC. FIG. 3B shows the relative expression of GalR1 mRNA in CK19, CK8, and desmin-immunolabeled cells. FIG. 3C shows the relative expression of GalR2 mRNA in CK19-, CK8-, and desmin-immunolabeled cells. *: CK8 or desmin vs CK19; #: Mdr2KO vs FVBN; @: male vs female; N=4, p<0.05.

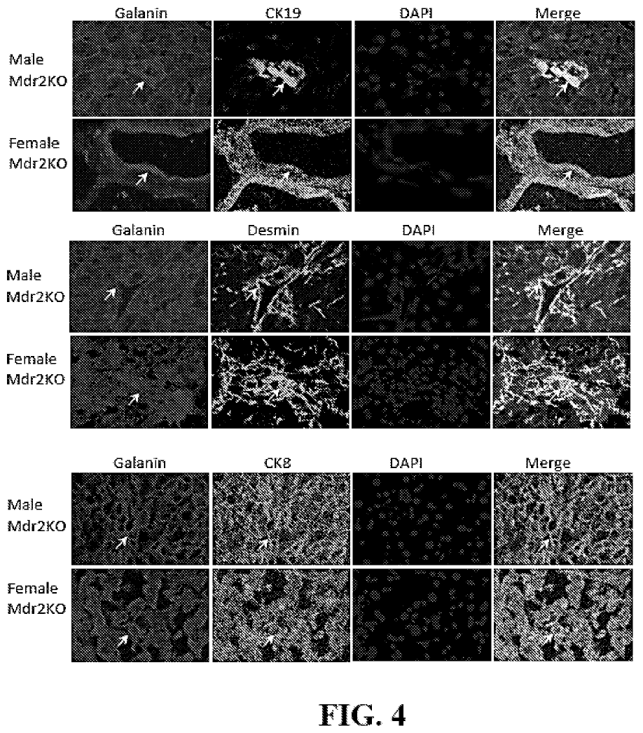

The distribution of Gal in various cells within the liver was also investigated by IF and confocal microscopy (FIG. 2A-C and FIG. 4). In FVBN mice, the hepatic Gal was only slightly detected (FIG. 2A-C). An overall greater amount of Gal was found by IF in liver tissue of male and female Mdr2KO mice than in FVBN controls (FIG. 2A-C). Images at higher magnification proved robust overlay of Gal staining with cholangiocytes, and in less extent with hepatocytes and HSC (FIG. 4). Thus, co-localization of Gal with CK19, indicated that galanin was abundantly present in cholangiocytes, inside the cytoplasm (FIG. 4). Galanin appeared also to be colocalized with desmin, a marker of HSC in male and female Mdr2KO mice (FIG. 4); however, Gal was distributed along the cells on their plasma membranes. Images of Gal colocalization with CK8, a marker of hepatocytes, demonstrate that a significant amount of galanin is associated with these cells, and Gal is concentrated on the plasma membrane of hepatocytes (FIG. 4).

Referring to FIG. 4, liver frozen sections from 2 months old, male and female, FVBN and Mdr2KO mice were immunolabeled with dual fluorescent flurophores and imaged by confocal microscopy in order to determine whether galanin peptide is localized in cholangiocytes (CK19), HSC (desmin), or hepatocytes (CK8).

4. Galanin Increases Intrahepatic Biliary Mass (IBDM) and Cholangiocyte Proliferation in MDR2KO and FVBN Mice

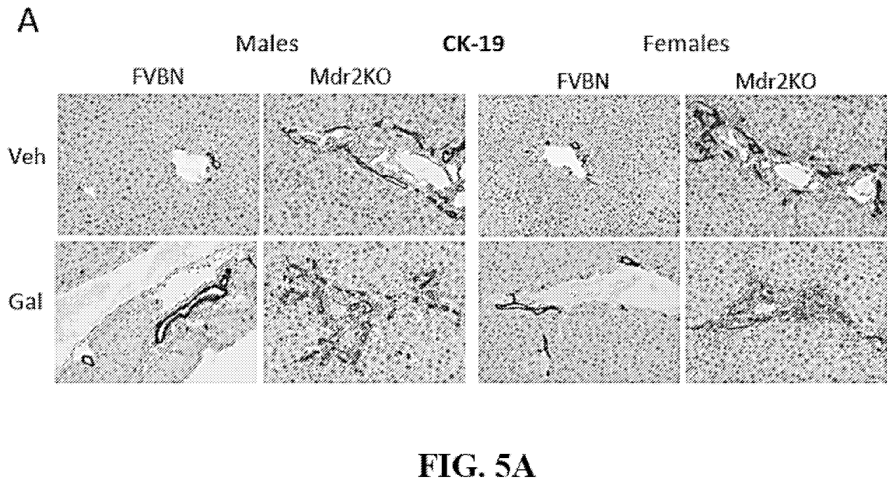

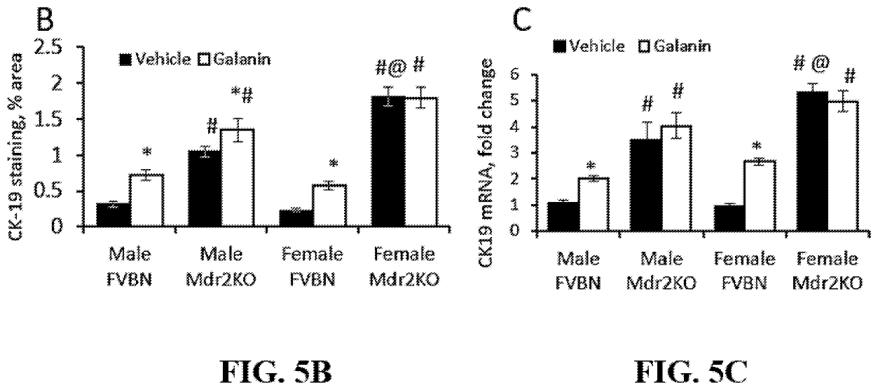

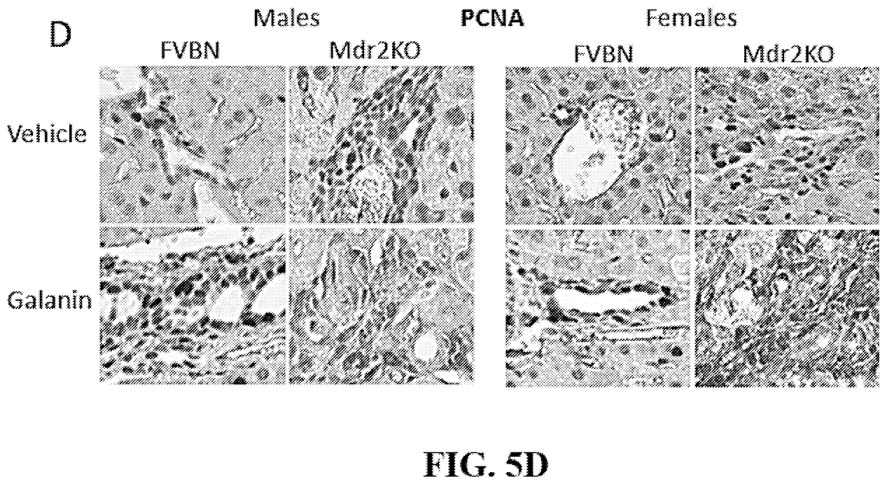

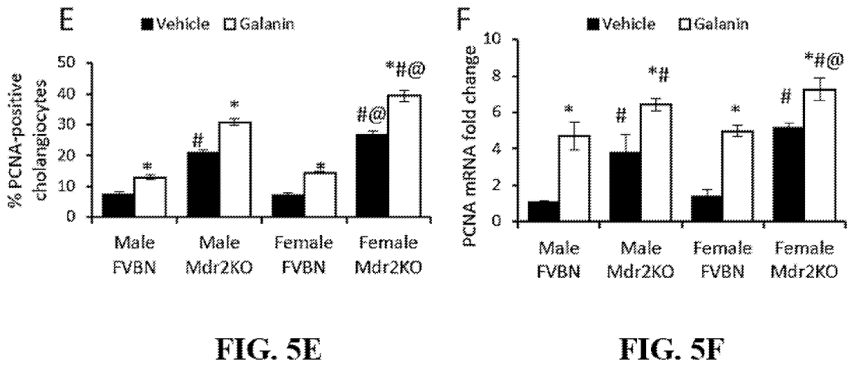

In order to assess the effect of Gal on cholangiocyte proliferation, CK19 was examined, a marker for cholangiocytes in liver sections of 2 months old, male and female Mdr2KO and FVBN mice which were treated with vehicle or Gal as described above. Cholangiocytes were detected by IHC and were quantified by image analysis (FIG. 5A and FIG. 5B), which demonstrated an increased IBDM in FVBN mice treated with galanin than in non-treated controls. In Mdr2KO mice which exhibit enlarged intrahepatic biliary ducts and have a high concentration of endogenous Gal, additional administration of galanin has increased IBDM only slightly in males but not in females Mdr2KO mice. Interestingly, at mRNA level, CK19 expression was significantly greater in Mdr2KO males and females (FIG. 5C) but this was not found at protein level, suggesting that regulatory mechanisms acting at protein translation or degradation prevent further increase of this protein.

Since PCNA is a marker of cell proliferation, the percentage of cholangiocytes which express PCNA was measured in males and females FVBN and Mdr2KO mice treated with vehicle or Gal (FIG. 5D and FIG. 5F), as well as the level of PCNA mRNA in livers of these groups of mice. The percentage of PCNA-expressing cholangiocytes was significantly increased in FVBN and Mdr2KO mice upon treatment with Gal. The PCNA mRNA was also enhanced by treatment of FVBN and Mdr2KO mice with Gal. Without wishing to be bound by theory, these data demonstrate that Gal has a role in cholangiocyte proliferation.

Referring to FIG. 5A-F, two months old, male and female, Mdr2KO and FVBN mice were treated with galanin or vehicle, as indicated elsewhere herein. Liver sections were processed for IHC staining of cholangiocyte marker CK19. FIG. 5A shows IHC images of CK19. FIG. 5B shows quantification of CK19 protein by image analysis. FIG. 5C shows quantification of CK19 mRNA expression by RT-qPCR. FIG. 5D shows IHC images of PCNA. FIG. 5E shows quantification of PCNA protein by IHC image analysis, by measuring the percentage of cholangiocytes which are PCNA-positive. FIG. 5F shows the relative expression of PCNA mRNA in liver tissue was quantified by qPCR. *: galanin vs vehicle; #: Mdr2KO vs FVBN; @: male vs female; N=5, p<0.05.

5. Galanin Exacerbates Fibrosis in FVBN and Mdr2Ko Mice

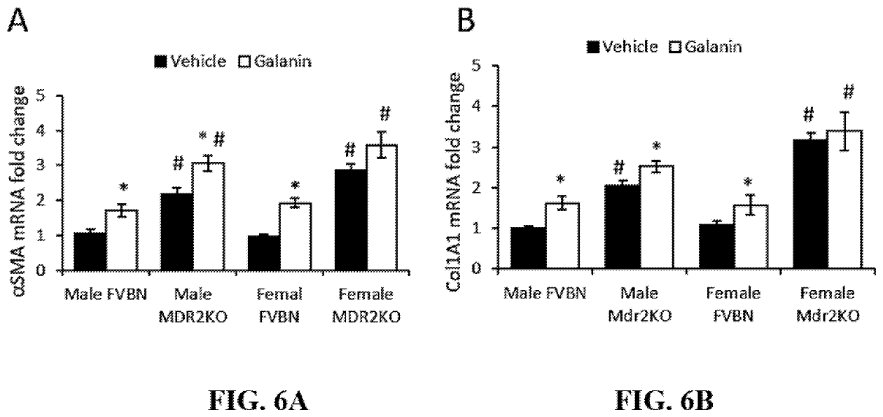

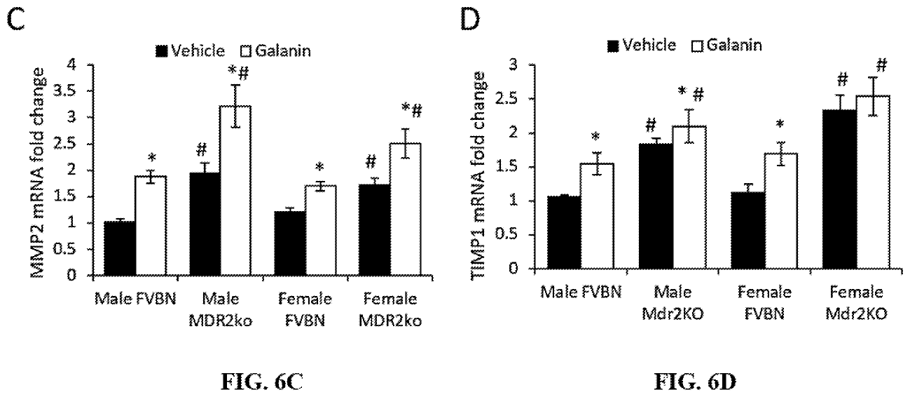

The mRNA expression of several markers of hepatic fibrosis including aSMA, Col1A1, MMP2, and TIMP1, were measured in livers of FVBN and Mdr2KO mice treated with vehicle or Gal (FIG. 6A-C). All of these markers were increased by Gal treatment, in FVBN and Mdr2KO mice, indicating that Gal is able to modulate the expression of these genes with roles in hepatic fibrosis.

Referring to FIG. 6A-D, the expression of hepatic fibrosis genes aSMA (FIG. 6A), Col1A1 (FIG. 6B), MMP2 (FIG. 6C), and TIMP1 (FIG. 6D) at mRNA level was assessed by RT-qPCR in liver tissue from male and female, FVBN and Mdr2KO mice, two months old, treated with vehicle or galanin. N=4, p<0.05.

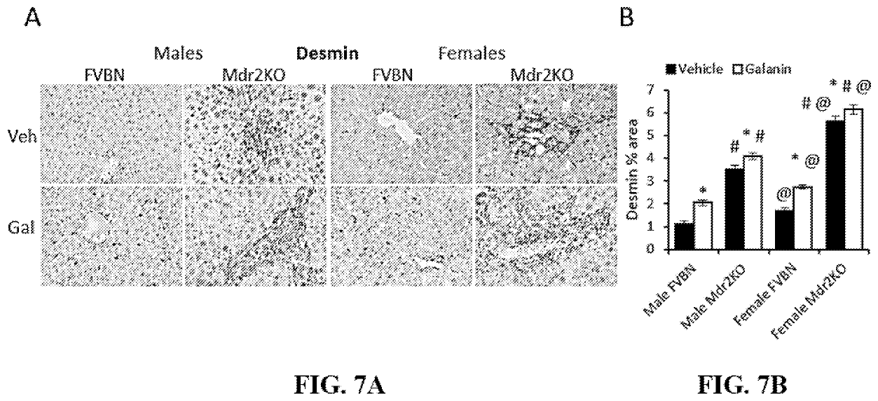

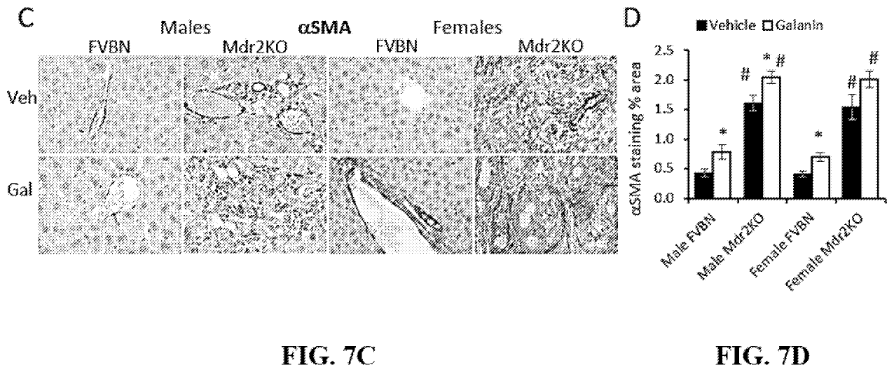

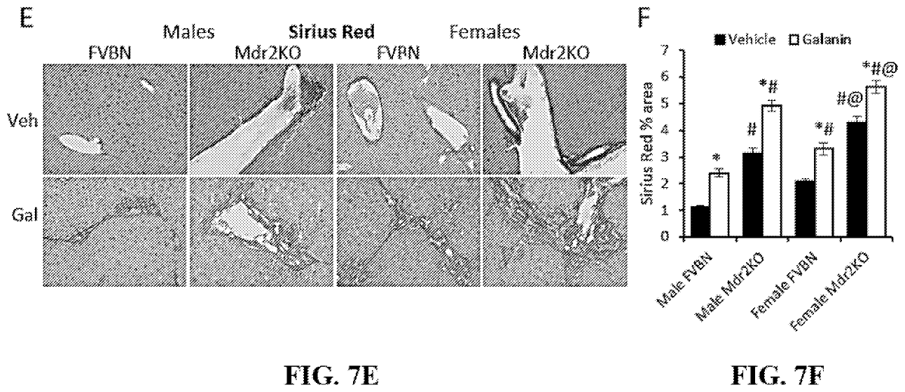

At the protein level, several markers of liver fibrogenesis were assessed, including desmin, aSMA and collagen types I and III (FIG. 7A-F). Desmin is expressed in all HSC, including quiescent, activated, and inactive cells (Puche et al. (2013) Compr. Physiol. 3: 1473-92). Images of desmin IHC staining, as well as quantifications by image analysis, indicate that there is a large increase of desmin expressing cells in MDR2KO mice as compared to FVBN controls, with female Mdr2KO mice having more HSC than male Mdr2KO mice (FIG. 7A and FIG. 7B). The treatment with Gal had a significant effect especially in FVBN mice increasing desmin-positive cells, in FVBN more than in Mdr2KO mice (FIG. 7A and FIG. 7B). The females expressed more desmin than males in livers of FVBN and Mdr2KO mice treated with Gal. The marker of activated HSC, aSMA, was more abundant in liver sections from FVBN and MDR2KO mice treated with Gal, than in vehicle-treated controls (FIG. 7C and FIG. 7D). Sirius Red staining of collagen types I and III, also demonstrated that Gal increased hepatic fibrosis in FVBN and Mdr2KO mice (FIG. 7E and FIG. 7F). The effect of Gal on enhanced expression of these fibrogenesis markers was greater in FVBN than in Mdr2Ko mice, as observed for desmin and aSMA. However, in the case of collagens I and II, the exogenously added galanin had a significant stimulatory effect even in the Mdr2KO mice, indicating that Gal differentially affects the expression of various genes involved in fibrosis progression (FIG. 7E and FIG. 7F).

Referring to FIG. 7A-F, markers of fibrosis such as aSMA, Col1A1, and desmin were measured in liver of 2 months old male and female, FVBN and Mdr2KO mice at protein level, by IHC. FIG. 7A shows IHC staining of desmin in liver tissue of male and female FVBN and Mdr2KO mice when treated with vehicle or galanin. FIG. 7B shows the quantification by image analysis of desmin IHC and graph showing area percentage of desmin staining. FIG. 7C shows representative images of IHC staining of aSMA. FIG. 7D shows quantification of aSMA IHC staining by image analysis. FIG. 7E shows Sirius Red staining of collagen type I and III within extracellular matrix of the liver. FIG. 7F shows quantification of Sirius Red-stained areas in images described in FIG. 7E. *: Galanin vs vehicle; #: Mdr2KO vs FVBN; @: male vs female; N=5, p<0.05.

6. GalR1 Vivo-Morpholino Treatment of Mdr2Ko Mice Reduces IBDM and Liver Fibrosis

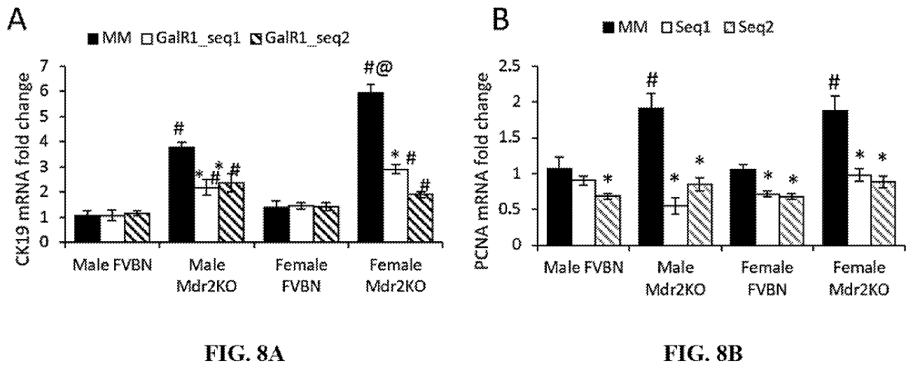

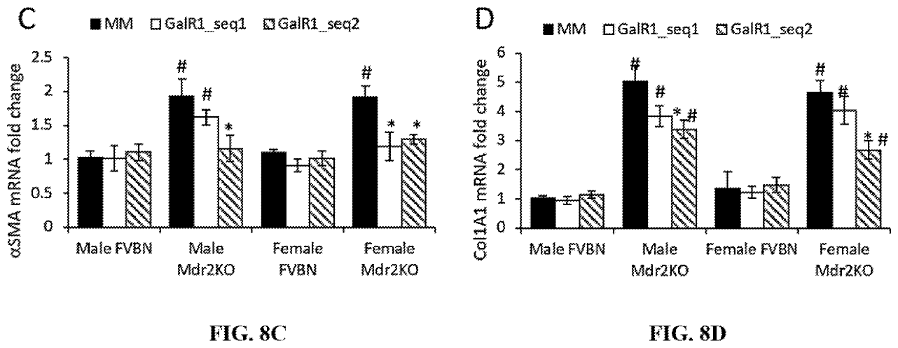

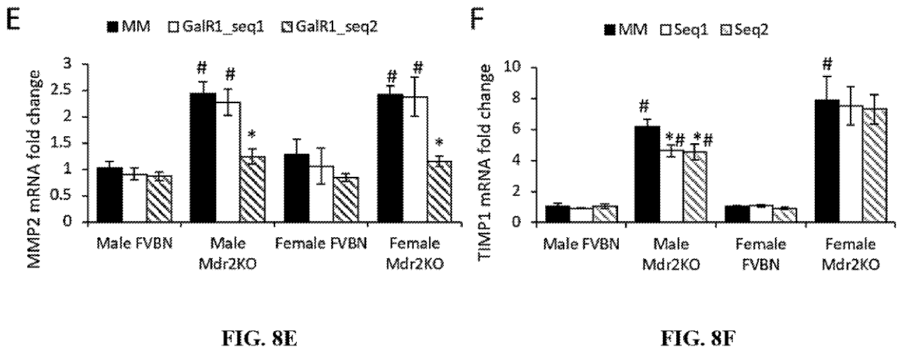

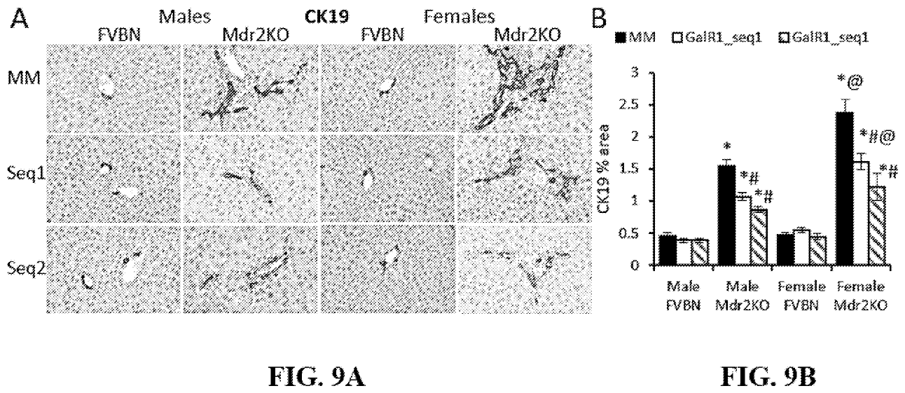

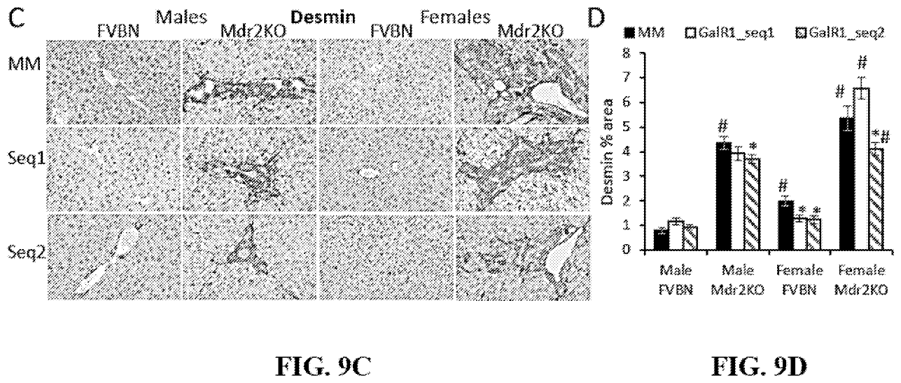

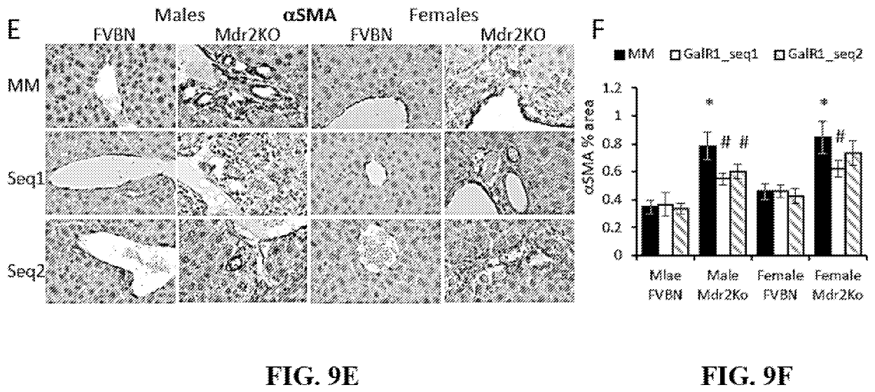

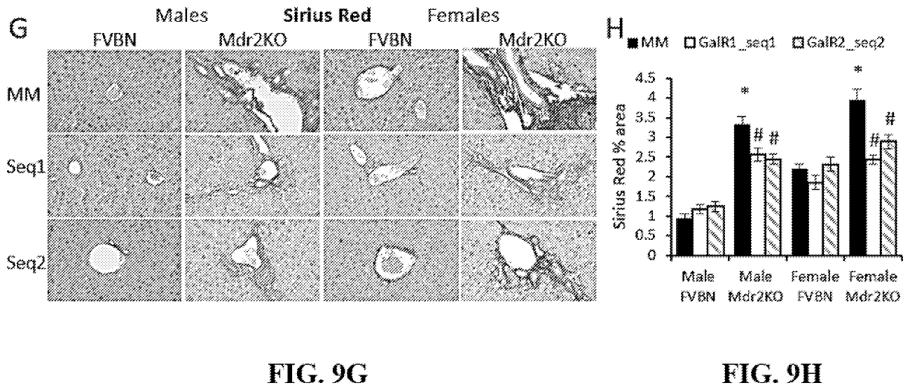

FVBN and Mdr2KO mice were administered GalR1-specific vivo morpholino sequences, in an attempt to reduce expression of this particular galanin receptor, which is expressed in intrahepatic cholangiocytes. The IBDM was then assessed by qPCR (FIG. 8A) and IHC of cholangiocyte marker CK19 (FIG. 9A and FIG. 9B). Both GalR1 vivo morpholino sequences significantly reduced CK19 mRNAs and CK19 protein in male and female Mdr2KO mice as compared to mismatch vivo morpholino-treated mice (FIG. 8A, FIG. 9A, and FIG. 9B). The PCNA marker of cell proliferation, was significantly reduced in Mdr2KO mice treated with GalR1-specific vivo morpholino as compared to Mdr2KO mice treated with the mismatch control sequence (FIG. 8B).

The expression of fibrosis markers aSMA, Col1A1, MMP2 and TIMP1 at mRNA level, was downregulated in male and female Mdr2KO mice treated with GalR1-specific vivo morpholino, as compared to mismatch-treated controls (FIG. 8C-F). However, the inhibitory effect of GalR1 vivo morpholino sequences was smaller for aSMA, Col1A1 fibrotic markers than for CK19 and PCNA markers of cholangiocyte proliferation (FIG. 8C-F). The protein expression levels of desmin (FIG. 9C and FIG. 9D), aSMA (FIG. 9E and FIG. 9F) and collagens I and III (FIG. 9G and FIG. 9H) were also measured, and indicated a significant decrease of these fibrotic markers in Mdr2KO mice treated with GalR1 vivo morpholino as compared to mismatch. Desmin was found to be the least affected by GalR1-vivo morpholinos (FIG. 9D). In conclusion, the effect of GalR1 vivo morpholino sequences was smaller for fibrosis markers than for cholangiocyte marker, suggesting that GalR1 is critical in galanin-induced biliary hyperplasia, and in a less extent, in Gal-induced fibrogenesis.

Referring to FIG. 8A-F, expression of CK19 (FIG. 8A), PCNA (FIG. 8B), aSMA (FIG. 8C), Col1A1 (FIG. 8D), MMP2 (FIG. 8E) and TIMP1 (FIG. 8F) mRNAs, was assessed by RT-qPCR in livers of FVBN and Mdr2KO mice treated with GalR1-specific vivo morpholino sequences (GalR1 seq1, GalR1 seq2) or negative control mismatch sequence (MM). N=4, p<0.05.

Referring to FIG. 9A-H, two months old male and female Mdr2KO mice and FVBN controls, were treated with GalR1 vivo morpholino sequences 1 (GalR1-seq1) or 2 (GalR1 seq2), or with mispair negative control sequence (MM), and then tested for IBDM hyperplasia and hepatic fibrosis markers, by IHC. FIG. 9A shows representative images of CK19 IHC in livers of mice treated with MM, GalR1 seq1, GalR1 seq2. FIG. 9B shows quantification of CK19 IHC by image analysis. FIG. 9C shows images of desmin IHC in liver from FVBN and Mdr2KO mice treated with vivo morpholino sequences. FIG. 9D shows quantification of desmin expression by image analysis. FIG. 9E shows representative images of aSMA IHC. FIG. 9F shows quantification of aSMA expression in livers of mice treated with GalR1 vivo morpholino vs. MM control, by image analysis. FIG. 9G shows representative images of Sirius Red staining of liver sections from mice treated with vivo morpholinos for GalR1, or MM. FIG. 9H shows quantifications of Sirius Red staining of collagen I and II from images in FIG. 9G. *: GalR1 vivo morpholino vs vehicle; #: Mdr2KO vs FVBN; @: male vs female; N=4, p<0.05.

7. GALR2-Specific Antagonist M871 Alleviates Liver Fibrosis in Mdr2Ko Mice without Changing the IBDM

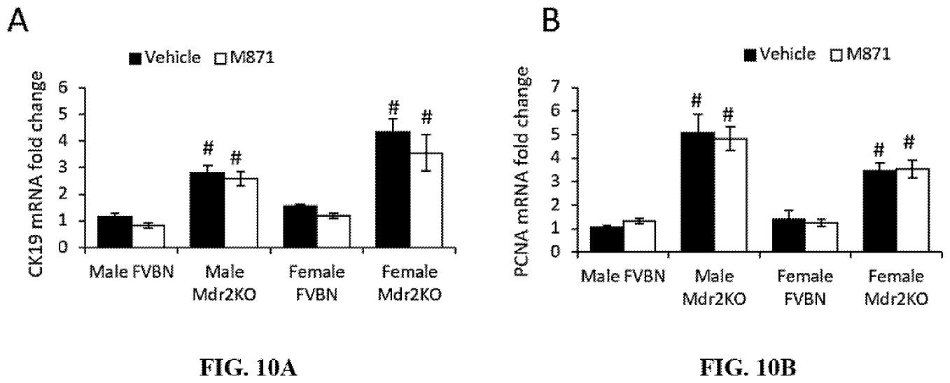

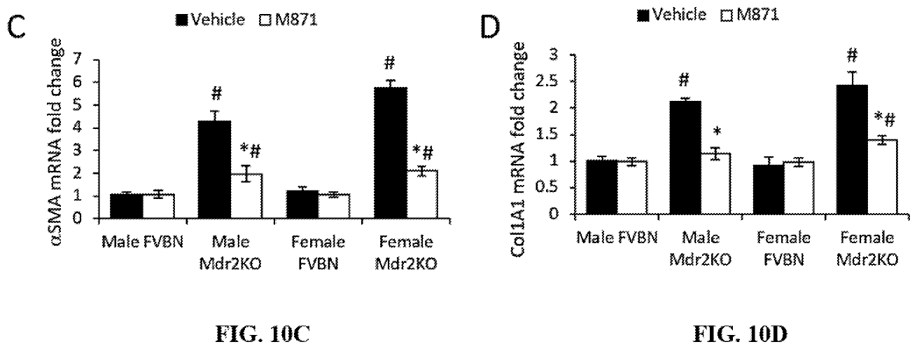

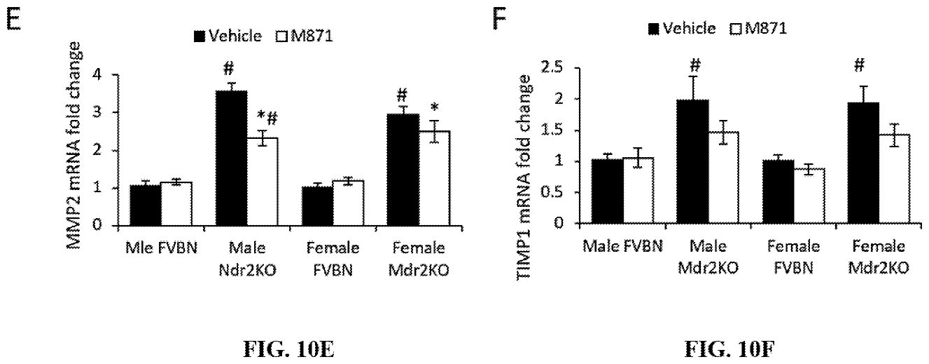

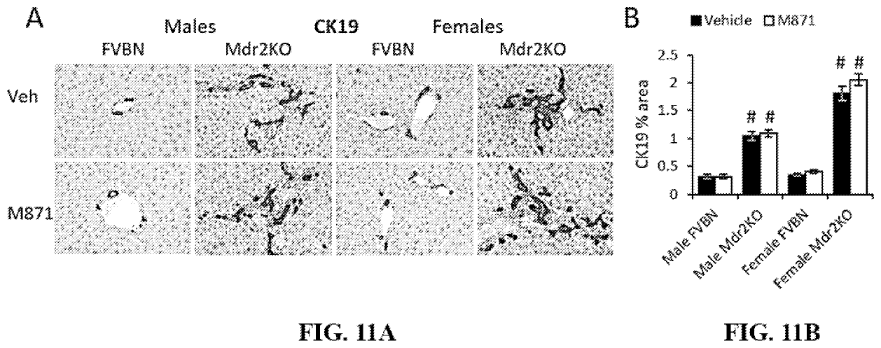

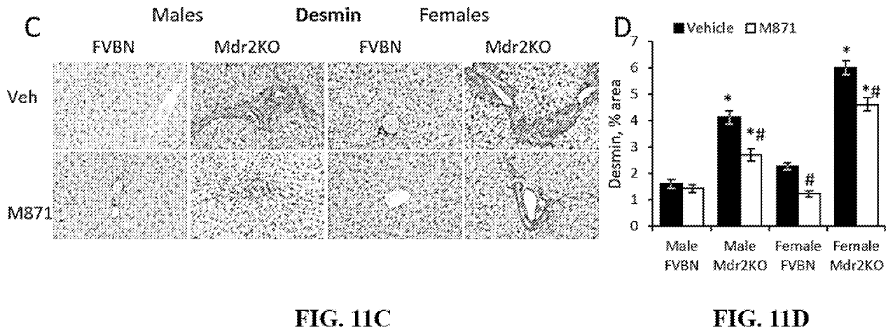

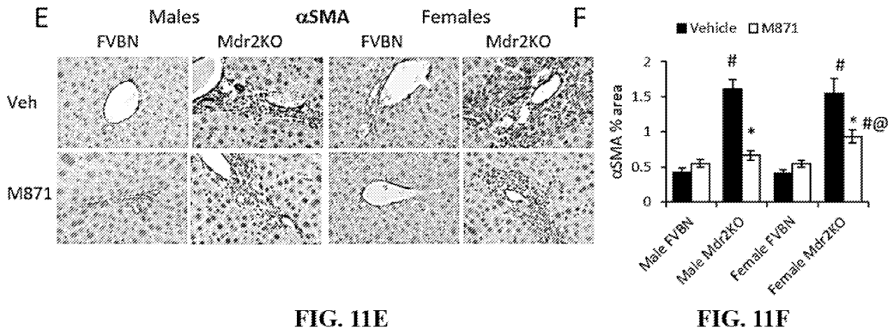

In order to assess the role of GalR2 in Gal-induced cholangiocyte proliferation, male and female Mdr2KO and FVBN mice were treated with vehicle or M871, a GalR2-specific antagonist. The data indicated that M871 did not affect CK19, nor PCNA mRNA expression in Mdr2KO mice, males or females (FIG. 10A and FIG. 10B). The same results were obtained by assessing CK19 protein expression by IHC (FIG. 11A and FIG. 11B).

The expression of several markers of fibrosis at was tested by qPCR and IHC, in mice treated with M871 versus mice treated with vehicle. The data showed that aSMA, Col1A1, MMP2, and TIMP1 mRNAs were downregulated in Mdr2KO mice treated with M871 (FIG. 10C-F). At protein level, desmin, as determined by IHC, was significantly decreased as a result of Mdr2KO mice treatment with GalR2 antagonist M871 (FIG. 11C and FIG. 11D). The assessment of aSMA by IHC (FIG. 11E and FIG. 11F), and of collagen types I and III by Sirius Red staining (FIG. 11G), confirmed that M871 was able to reduce hepatic fibrosis in Mdr2KO mice. Without wishing to be bound by theory, the results indicate that inhibition of GalR2 is conducive to a significant reduction of hepatic fibrosis markers in Mdr2KO mice as compared to mice treated with vehicle only, while not having any effect on biliary hyperplasia.

Referring to FIG. 10A-F, expression of CK19 (FIG. 10A), PCNA (FIG. 10B), aSMA (FIG. 10C), Col1A1 (FIG. 10D), MMP2 (FIG. 10E) and TIMP1 (FIG. 10F) mRNAs, was assessed by RT-qPCR in livers of FVBN and Mdr2KO mice treated with vehicle or M871. N=4, p<0.05.

Referring to FIG. 11A-H, two months old male and female Mdr2KO and FVBN mice were treated with M871, a GalR2-specific antagonist, and tested for IBDM hyperplasia and hepatic fibrosis markers. FIG. 11A shows the expression of CK19 marker of cholangiocytes, at mRNA level was quantified by qPCR in liver tissue from mice treated with vehicle or M871. FIG. 11B shows quantification of CK19 protein by image analysis in liver sections processed by IHC. FIG. 11C shows representative images of CK19 IHC, in liver from mice treated with vehicle or M871. FIG. 11D shows the relative expression of aSMA mRNA in liver of mice treated with vehicle or M871. FIG. 11E shows the relative expression of Col1A1 mRNA in liver of mice treated with vehicle or M871. FIG. 11F shows representative images of Sirius Red stained deposits of collagen I and III in liver of mice treated with vehicle or M871. FIG. 11G shows image analysis and quantification of Sirius Red percent area in liver sections from mice treated with vehicle or M871. *: M871 vs vehicle; #: Mdr2KO vs FVBN; @: male vs female; N=4, p<0.05.

8. An Antagonist of Both GalR1 and GalR2 Reduces Cholangiocyte Proliferation and Hepatic Fibrosis in Mdr2Ko Mice

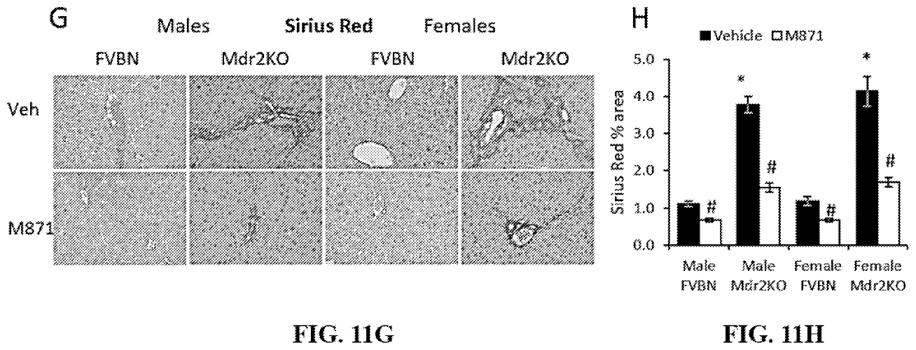

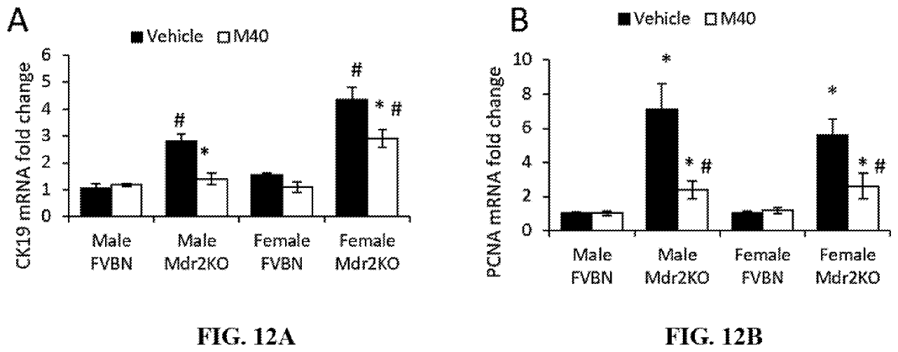

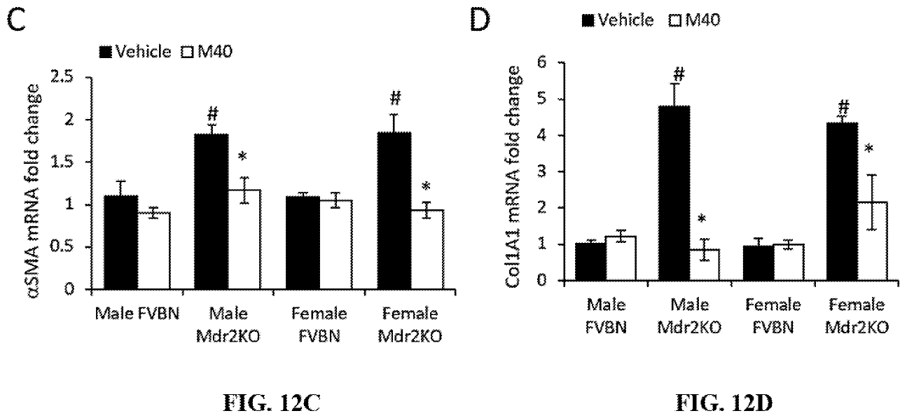

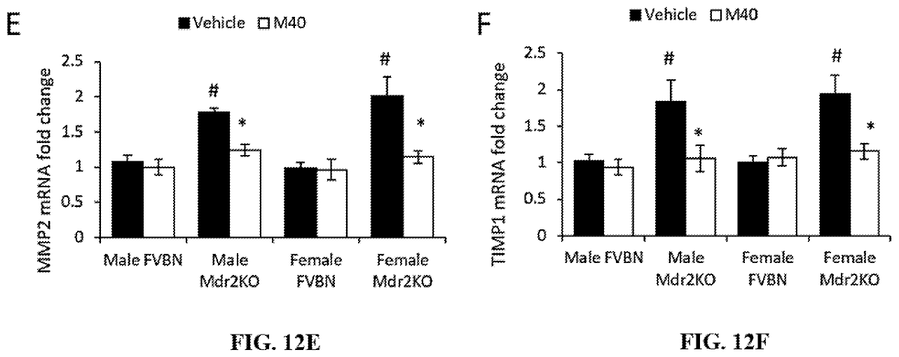

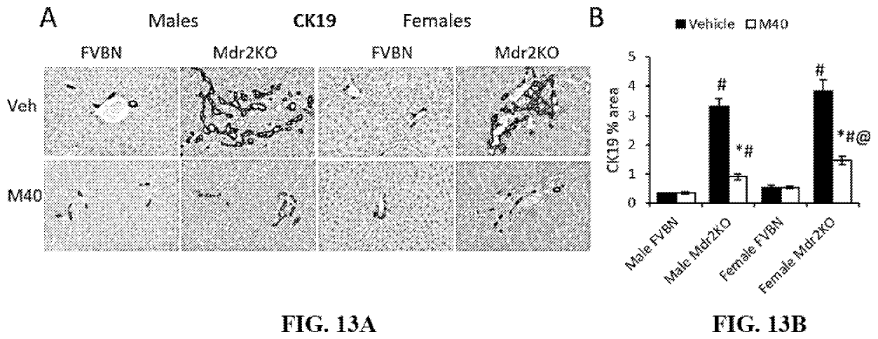

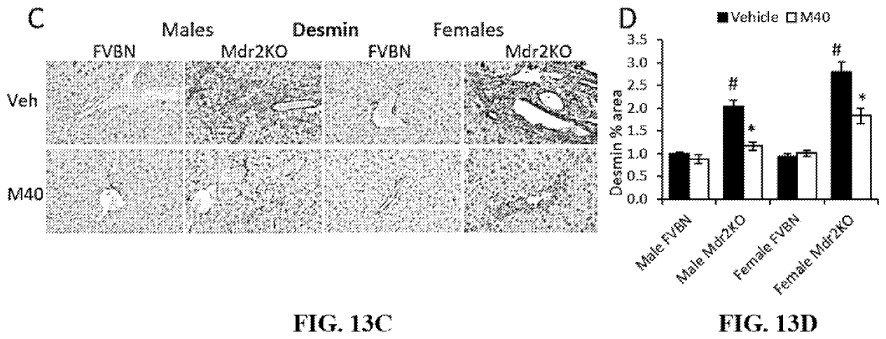

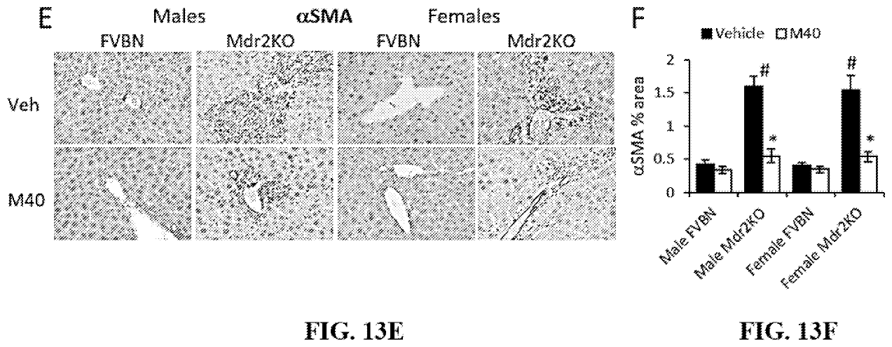

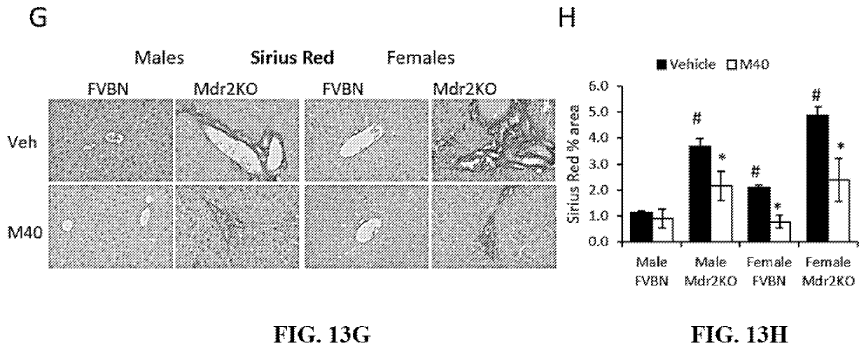

M40, a nonspecific antagonist of GalR1 and GalR2, was also used to test the role of these receptors on Gal-induced biliary hyperplasia and hepatic fibrosis. M40 treatment caused a significant downregulation of CK19 and PCNA mRNAs in Mdr2KO mice (FIG. 12A and FIG. 12B). Further experiments to determine changes in CK19 protein expression by IHC, showed that treatment with M40 resulted in strong reduction of IBDM in Mdr2KO mice (FIG. 13A and FIG. 13B) when compared to MDR2KO mice treated with vehicle.

Expression of fibrogenesis markers including aSMA, Col1A1, MMP2, and TIMP1 at mRNA level was also downregulated in Mdr2KO mice treated with M40 (FIG. 12C-F). Protein expression of fibrosis-related genes including desmin (FIG. 13C and FIG. 13D), aSMA (FIG. 8E and FIG. 13F) and of collagens I and III (FIG. 13G and FIG. 13H), confirmed that M40 treatment of Mdr2KO mice resulted in a significant reduction of hepatic fibrosis in these mice. Without wishing to be bound by theory, the results indicate that M40 antagonist decreases the effects of Gal through both GalR1 and GalR2, decreasing both cholangiocyte proliferation and hepatic fibrogenesis in Mdr2KO mice.

Referring to FIG. 12A-F, expression of CK19 (FIG. 12A), PCNA (FIG. 12B), aSMA (FIG. 12C), Col1A1 (FIG. 12D), MMP2 (FIG. 12E), and TIMP1 (FIG. 12F) mRNAs, was assessed by RT-qPCR in livers of FVBN and Mdr2KO mice treated with vehicle or M40. N=4, p<0.05.

Referring to FIG. 13A-H, two months old male and female Mdr2KO and FVBN mice were treated with M40, a non-specific antagonist of GalR1 and GalR2, and then tested for IBDM hyperplasia and hepatic fibrosis markers. FIG. 13A shows representative images of CK19 IHC in liver samples of mice treated with vehicle or M40. FIG. 13B shows image analysis of CK19 protein as detected by IHC in liver of mice treated with vehicle or M40. FIG. 13C shows images of desmin IHC in mice treated with vehicle or M40 and the relative expression of CK19 cholangiocyte marker mRNA in liver of mice treated with vehicle or M40. FIG. 13D shows quantification of desmin expression in liver tissue of mice treated with vehicle or M40, by image analysis. FIG. 13E shows IHC images of aSMA in liver of mice treated with vehicle or M40. FIG. 13F shows quantification of aSMA expression based on image analysis. FIG. 13G shows images of Sirius Red staining of collagen I and III in liver of mice treated with vehicle or M40. FIG. 13H shows quantification of Sirius Red-stained collagen I and III in liver of mice treated with vehicle or M40. *: M40 vs vehicle; #: Mdr2KO vs FVBN; @: male vs female; N=4, p<0.05.

9. Antagonists of Gal Receptors Reduce Systemic Gal and Liver Enzymes in Mdr2Ko Mice

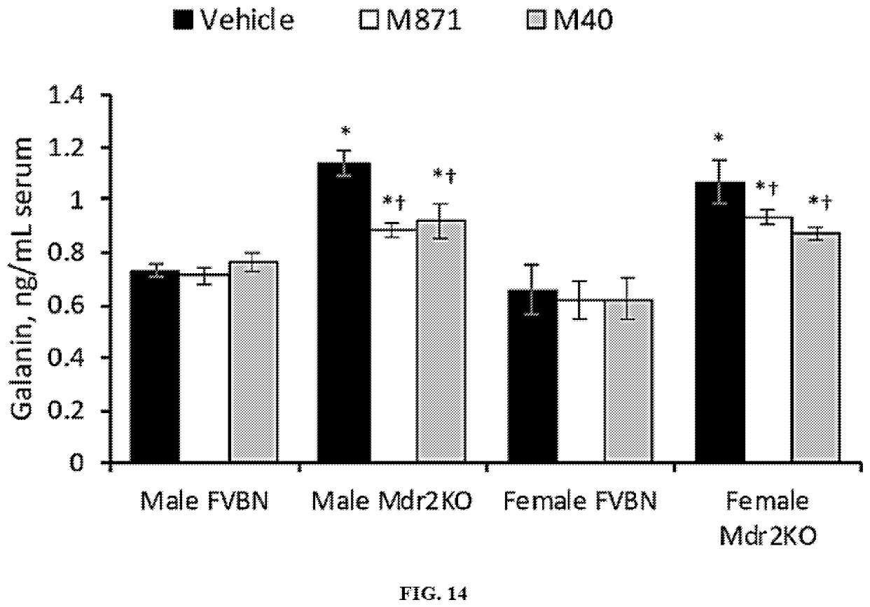

Serum concentrations of Gal were measured in FVBN and Mdr2KO mice that had been treated with M871 or M40 versus vehicle only (FIG. 14). Without wishing to be bound by theory, the results suggest that these antagonists of Gal receptors caused a modest but significant alleviation of the abnormally high levels of Gal in Mdr2KO mice. As shown in Table 1, all tested liver enzymes were elevated in Mdr2KO mice as compared to FVBN controls. However, ALT, AST, and ALPK were reduced in Mdr2KO mice treated with GalR1 vivo morpholino, M871, or M40, as compared to vehicle-treated Mdr2KO mice.

Referring to FIG. 14, GalR1 and GalR2 antagonists alleviate serum level of Gal in Mdr2KO mice. Gal concentration in serum from FVBN and Mdr2KO mice treated with vehicle, M871 or M40 was assessed by enzyme immunoassay. n=4 ( ). *P<0.05 Mdr2KO versus FVBN mice; TP<0.05 M871/M40 versus vehicle.

Referring to Table 1, liver enzyme assessment in FVBN and Mdr2KO mice treated with GalR antagonists versus vehicle controls is shown. ALT, alanine aminotransferase; AST, aspartate aminotransferase; ALKP, alkaline phosphatase. GalR1 seq1 is the GalR1-specific vivo morpholino sequence 1. *, vs Mdr2KO+vehicle; N=3, p<0.05.

TABLE 1

| | Sample | | | |
|---|---|---|---|---|
| Enzyme | Vehicle | GalR1_seq1 | M871 | M40 |

ALT (U/L)

| | | | | |
|---|---|---|---|---|
| Male FVBN. | 270 ± 11.54 | 236 ± 38.44 | 242 ± 32.14 | 373 ± 49.10 |
| Male Mdr2KO | 1007 ± 137.75 | 736 ± 129.91 | 370 ± 64.29* | 633 ± 113.48* |
| Female FVBN | 307 ± 85.70 | 306 ± 119.77 | 270 ± 11.55 | 163 ± 40.96 |
| Female Mdr2KO | 1150 ± 135.77 | 736 ± 129.91 | 953 ± 103.98 | 983 ± 169.05 |

AST (U/L)

| | | | | |
|---|---|---|---|---|
| Male FVBN. | 340 ± 28.87 | 310 ± 101.49 | 238 ± 87.62 | 410 ± 70.95 |
| Male Mdr2KO | 967 ± 41.77 | 807 ± 79.65 | 583 ± 131.32* | 626 ± 88.07* |
| Female FVBN | 293 ± 107.13 | 523 ± 208.99 | 427 ± 129.81 | 459 ± 114.89 |
| Female Mdr2KO | 1210 ± 202.32 | 1160 ± 138.92 | 863 ± 116.67 | 950 ± 40.41 |

ALKP (U/L)

| | | | | |
|---|---|---|---|---|
| Male FVBN. | 99 ± 0.67 | 100 ± 0.11 | 107 ± 12.02 | 106 ± 6.67 |
| Male Mdr2KO | 193 ± 21.86 | 117 ± 8.82 | 103 ± 3.33* | 100 ± 0.00* |
| Female FVBN | 97 ± 3.33 | 100 ± 0.00 | 98 ± 3.84 | 97 ± 3.33 |
| Female Mdr2KO | 200 ± 55.08 | 150 ± 20.82 | 193 ± 18.56 | 193 ± 69.84 |

10. Non-Hepatic Effects of Gal Receptor Antagonists in FVBN and Mdr2Ko Mice

To investigate possible adverse effects of GalR antagonists on vital organs other than the liver, blood biomarkers of the heart (creatine kinase, CK) and kidneys (creatinine, CREA; blood urea nitrogen, BUN) were investigated in FVBN and Mdr2KO mice when treated with GalR1 vivo morpholino, M871, and M40 as compared to vehicle-treated mice. Without wishing to be bound by theory, the results indicated that the suppression of Gal receptors had no effects on the heart or kidneys (Table 2).

Referring to Table 2, non-hepatic effects of GalR1 vivo morpholino, M871, and M40 Gal receptor antagonists on FVBN mice are shown. Several serum biomarkers including creatine kinase (CK) for heart, creatinine (CREA) and blood urea nitrogen (BUN) for kidney, were assessed by using IDEXX assay system. Normal range: CK, 68-1070 U/L; CREA, 0.2-0.8 mg/mL; BUN, 18-29 mg/mL.

TABLE 2

| | Sample | | | |
|---|---|---|---|---|
| Enzyme | Vehicle | GalR1_seq1 | M871 | M40 |

CK (U/L)

| | | | | |
|---|---|---|---|---|
| Male FVBN. | 100.33 ± 1.45 | 99.33 ± 0.67 | 99.67 ± 0.88 | 190.00 ± 58.59 |
| Male Mdr2KO | 100.33 ± 0.33 | 130.00 ± 30.01 | 230.00 ± 52.92 | 103.33 ± 3.33 |
| Female FVBN | 100.67 ± 1.20 | 106.67 ± 6.67 | 136.67 ± 36.67 | 151.30 ± 19.90 |
| Female Mdr2KO | 99.33 ± 1.45 | 110.01 ± 10.01 | 213.33 ± 103.49 | 163.33 ± 63.33 |

CREA (U/L)

| | | | | |
|---|---|---|---|---|
| Male FVBN. | 0.70 ± 0.054 | 0.66 ± 0.074 | 0.81 ± 0.063 | 0.77 ± 0.053 |
| Male Mdr2KO | 0.59 ± 0.071 | 0.77 ± 0.083 | 0.67 ± 0.042 | 0.57 ± 0.062 |
| Female FVBN | 0.67 ± 0.033 | 0.79 ± 0.051 | 0.79 ± 0.041 | 0.83 ± 0.077 |
| Female Mdr2KO | 0.49 ± 0.014 | 0.68 ± 0.062 | 0.68 ± 0.012 | 0.71 ± 0.058 |

BUN (U/L)

| | | | | |
|---|---|---|---|---|
| Male FVBN. | 27.57 ± 0.43 | 26.97 ± 0.48 | 26.97 ± 0.83 | 25.40 ± 1.47 |
| Male Mdr2KO | 24.33 ± 0.39 | 27.63 ± 0.37 | 23.04 ± 0.92 | 24.50 ± 1.63 |
| Female FVBN | 26.36 ± 0.70 | 24.83 ± 0.17 | 26.55 ± 1.67 | 22.05 ± 0.93 |
| Female Mdr2KO | 25.34 ± 0.45 | 26.43 ± 0.57 | 23.33 ± 0.49 | 26.11 ± 1.33 |

11. Effect of Galanin on Intrahepatic Biliary Mass (IBDM) and Hepatic Fibrosis

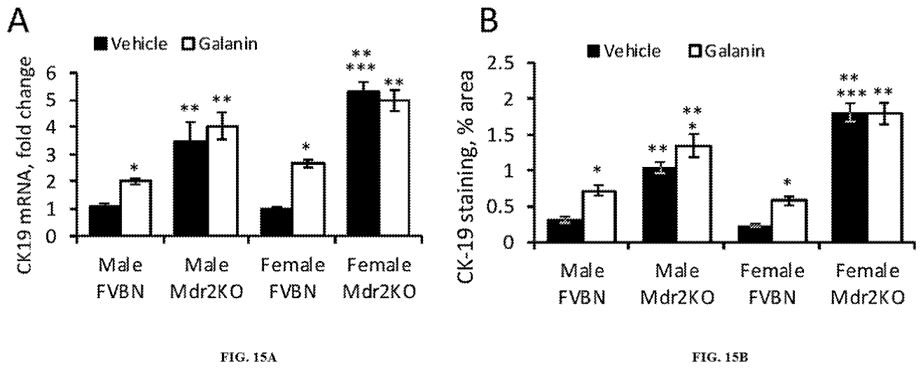

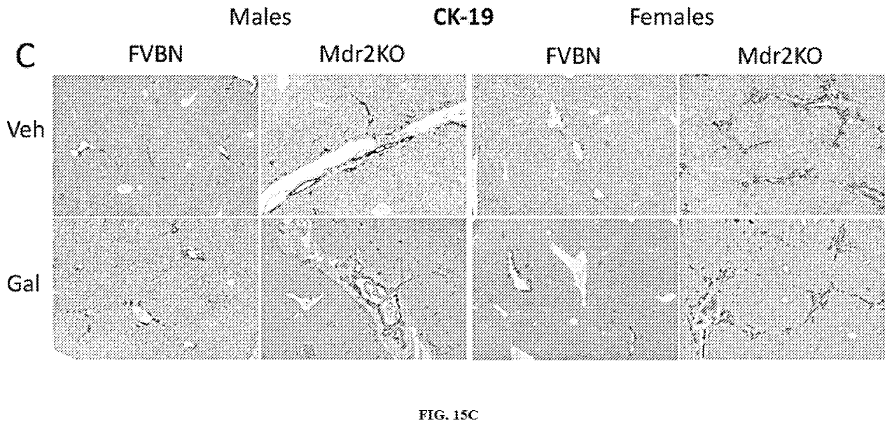

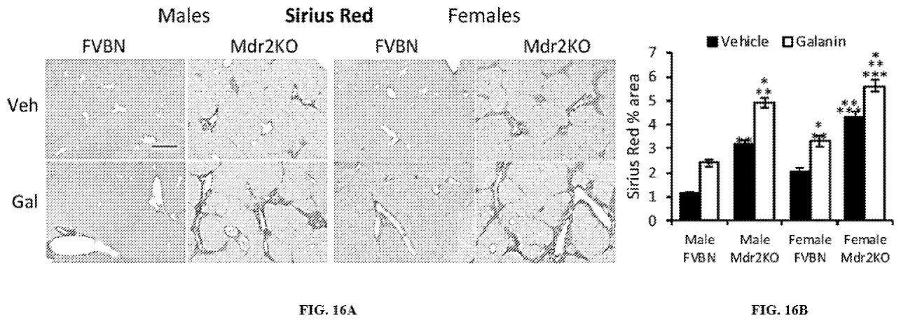

Mdr2KO and FVBN mice treated with vehicle or Gal were tested for the expression of CK19 (FIG. 15A-C) and fibrosis (FIG. 6A-C, FIG. 7A-D, FIG. 16A, and FIG. 16B) at mRNA and protein level. In Mdr2KO mice, Gal increased IBDM slightly in males but not in females, while a significant increase in CK19 mRNA and protein was detected in all FVBN mice treated with Gal over non-treated controls (FIG. 15A-C). Similarly, fibrosis biomarkers α-SMA, Col1A1, MMP2, and TIMP1 were increased more in FVBN than in Mdr2KO mice by Gal treatment (FIG. 6A-C, FIG. 7A-D, FIG. 16A, and FIG. 16B). Galanin differentially affected the expression of various genes involved in fibrosis progression. Thus, it increased desmin and aSMA in FVBN mice only while it stimulated expression of collagens I and III, in both FVBN and Mdr2KO mice.

Referring to FIG. 15A-C, liver from 2 month old Mdr2KO and FVBN mice treated with Gal or vehicle, were assayed for CK19 mRNA expression (FIG. 15A), and CK19 IHC (FIG. 15B and representative images in panel and quantifications in FIG. 15C). *Gal vs vehicle. **Mdr2KO vs FVBN. ***Male vs female; N=5, p<0.05.

Referring to FIG. 16A and FIG. 16B, expression of α-SMA, Col1A1, MMP2, and TIMP1 at mRNA level was assessed by RT-qPCR in liver from FVBN and Mdr2KO mice, two months old, treated with vehicle or galanin. See FIG. 6A-C and FIG. 7A-D. N=4, p<0.05. *Gal vs vehicle. **Mdr2KO vs FVBN. Liver sections from these mice were stained for desmin (FIG. 7A), α-SMA (FIG. 7C) by IHC, and for collagens I and III (FIG. 16A) with Sirius Red. Quantifications were performed by image analysis (FIG. 7B, FIG. 7D, and FIG. 16B). * Gal vs veh. **Mdr2KO vs FVBN. ***female vs male. N=5, p<0.05. Scale bar, 100 μm.

12. GalR1 Expression in Cholangiocytes In Vitro is Essential for Gal-Induced Activation of HSC

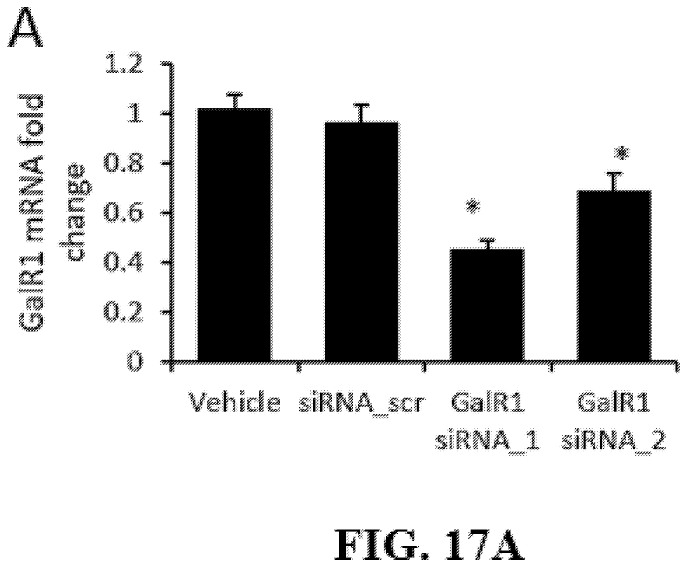

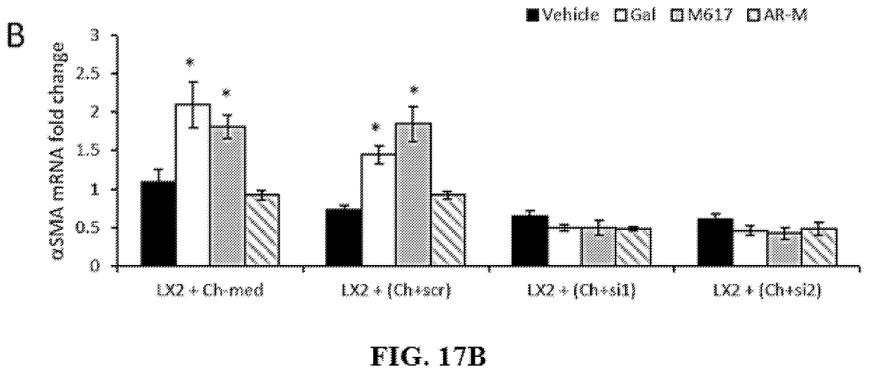

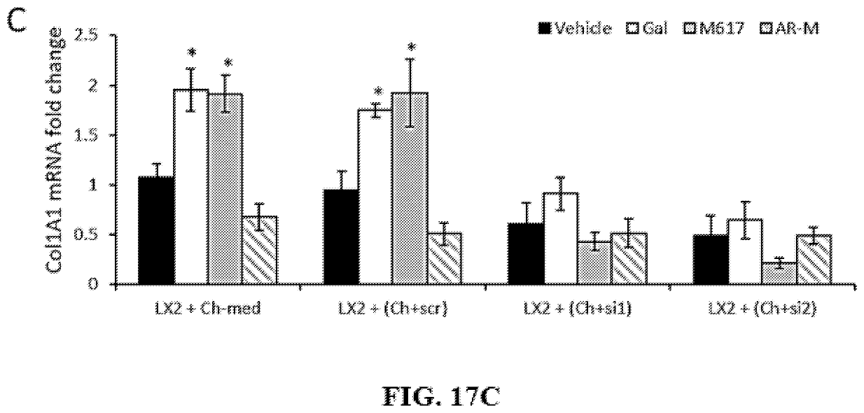

Mouse pooled cholangiocytes were transfected with two sequences of siRNA in order to knock down GalR1 transcription. The reduction of GalR1 expression in these cells was successful, as confirmed by GalR1 qPCR of RNA isolated from transfected cells and compared to control (scramble) siRNA. Data in FIG. 17A indicate that both siRNAs reduced GalR1 expression.

LX-2 cells were incubated with conditioned media from cholangiocytes in which GalR1 was knocked down with siRNA-1 and -2, versus negative control (cholangiocytes transfected with scr-siRNA), in the absence or presence of agonists specific to GalR1 (M617), GalR2 (AR-M) or both (Gal), and then the activation of LX-2 HSC was measured by qPCR for aSMA and Col1A1 (FIG. 17C and FIG. 17D). Both fibrosis markers were unregulated by Gal and M617 but not AR-M in positive controls, i.e., LX-2 cells treated with media from non-transfected cholangiocytes, or from cholangiocytes transfected with scr-siRNA. However, there was no increase in aSMA and Col1A1 mRNAs in LX-2 cells incubated with media from cholangiocytes transfected with GalR1 siRNAs-1, -2, and stimulated with Gal, M617 or AR-M treatments (FIG. 17C). Without wishing to be bound by theory, these results suggest that activation of LX-2 cells with cholangiocyte-conditioned media is possible only when GalR1 is functional in cholangiocytes.

Referring to FIG. 17A-C, two different sequences of siRNAs specific for GalR1 were used to transfect mouse pool cholangiocytes, vs negative control scr-siRNA. FIG. 17A shows GalR1 mRNA relative expression in cholangiocytes transfected with seq-1 and seq-2 vs non-transfected or scr-siRNA cholangiocytes. Changes in mRNA expression of aSMA (FIG. 17B) and Col1A1 (FIG. 17C) in LX-2 cells when incubated with conditioned media from non-transfected cholangiocytes, of cholangiocytes transfected with scr, seq-1, seq-2 siRNAs, plus vehicle, galanin, M617 or AR-M are also shown. *: galanin/M617/AR-M vs vehicle; #: media from cholangiocytes in which GalR1 is silenced with seq1 or seq2, vs media from cholangiocytes transfected with scr-siRNA negative control. N=4, p<0.05.

13. GalR2 is Critical for Lx-2 Cells Activation by Gal Directly or Via Cholangiocyte Conditioned Media

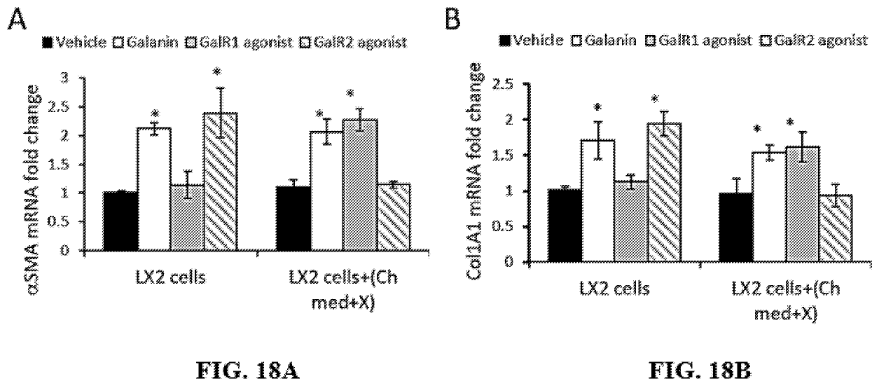

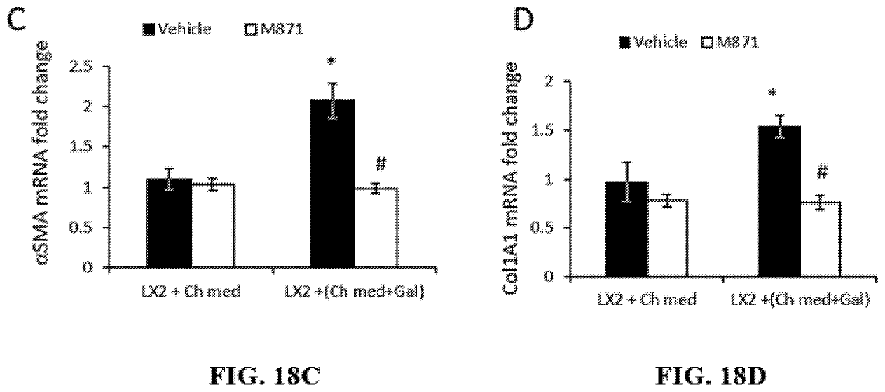

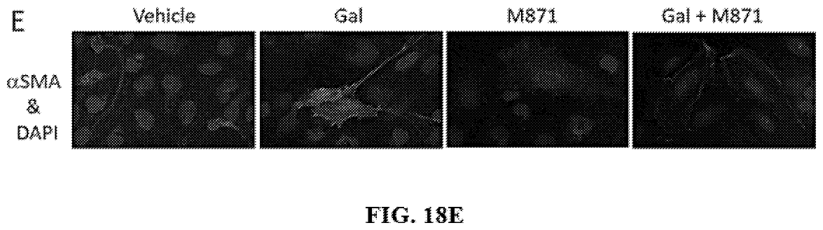

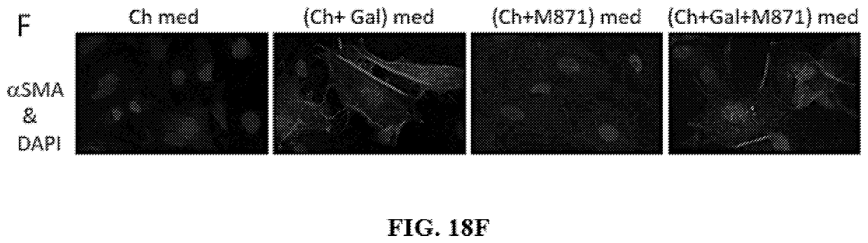

The role of GalR2 in the activation of LX-2 cells when stimulated directly or via cholangiocyte-conditioned media with Gal or specific agonists of GalR1 and GalR2 was investigated (FIG. 18A-F). Thus, aSMA and Col1A1 markers of fibrogenesis were assessed in LX-2 cells which were treated with vehicle, Gal, GalR1 agonist M617 or GalR2-specific agonist AR-M directly, or via cholangiocyte-conditioned media. Both markers were upregulated by Gal and GalR2-specific agonist when incubated directly with these peptides as compared to LX-2 cells treated with vehicle (FIG. 18A and FIG. 18B). However, aSMA and Col1A1 were upregulated in LX-2 cells when incubated with media from cholangiocytes stimulated by Gal and GalR1-specific agonist M617 but not by GalR2-specific agonist AR-M (FIG. 18A and FIG. 18B).

In another set of experiments, LX-2 cells were incubated with conditioned media from cholangiocytes treated with GalR2-specific antagonist M871, versus vehicle as negative control, then tested for αSMA and Col1A1 mRNA expression (FIG. 18C and FIG. 18D). The data demonstrated that stimulation of cholangiocytes by Gal was not sufficient for further activation of LX-2 cells when GalR2 was blocked in LX-2 cells by M871 antagonist. The upregulation of αSMA mRNA in LX-2 cells when treated with Gal in the absence or presence of GalR2 antagonist M871 directly or via cholangiocyte-conditioned media, was tested at protein level by IF (FIG. 18E and FIG. 18F). αSMA expression was increased when LX-2 cells were incubated with Gal or with media from Gal-treated cholangiocytes. In contrast, in the presence of M871, Gal did not induce αSMA expression in LX-2 cells directly, nor via cholangiocyte-conditioned media.

Referring to FIG. 18A-F, LX-2 cells were treated with vehicle, galanin, GalR1 agonist M617 or GalR2 agonist AR-M, or with conditioned media from cholangiocytes incubated with vehicle, galanin, M617, or AR-M. αSMA (FIG. 18A) and Col1A1 (FIG. 18B) mRNA expression was measured by RT-qPCR, for all these treatments. In a parallel set of experiments, αSMA (FIG. 18C) and Col1A1 (FIG. 18D) were quantified in LX-2 cells which were treated with cholangiocyte-conditioned media when cholangiocytes were incubated with vehicle or GalR2 antagonist M871. FIG. 18E shows immunofluorescence (IF) assay of αSMA protein in LX-2 cells when treated with vehicle, galanin (Gal), GalR2 antagonist M871, or Gal+M871. FIG. 18F shows representative results of an IF assay of αSMA in LX-2 cells when treated with conditioned media from cholangiocytes incubated with vehicle, galanin (Gal), GalR2 antagonist M871, or Gal+M871. *: cholangiocytes incubated with galanin vs cholangiocytes treated with vehicle; #: galanin, M617, Ar-M, M871 vs vehicle. N=4, p<0.05.

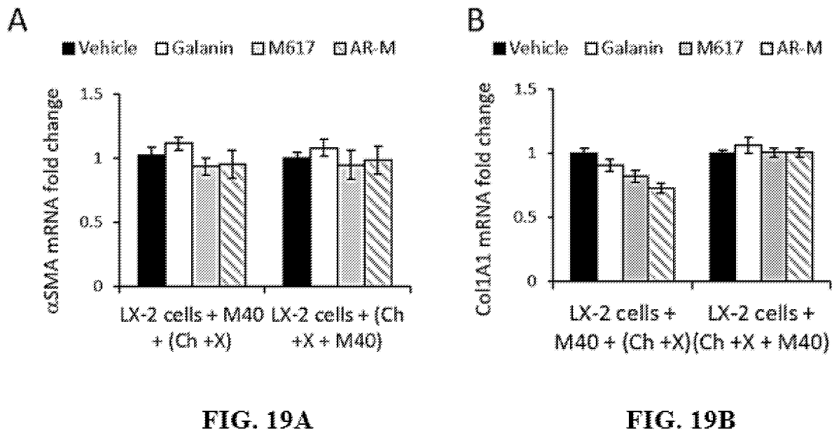

Finally, the effect of M40, a non-specific antagonist of both GalR1 and GalR2, was tested on cholangiocyte-mediated activation of LX-2 cells (FIG. 19A and FIG. 19B). None of the GalR1 or GalR2 agonists was able to induce upregulation of αSMA or Col1A1 when LX-2 cells were treated with M40 (FIG. 19A), or when cholangiocytes were treated with M40 (FIG. 19B).

Referring to FIG. 19A and FIG. 19B, αSMA (FIG. 19A) and Col1A1 (FIG. 19B) mRNAs were measured by RT-qPCR in LX-2 cells when treated with: i) M40 and media from cholangiocyte preincubated with an agonist X, where X=galanin, M617 or AR-M (LX-2 cells+M40+(Ch+X)); ii) media from cholangiocytes treated with an agonist (X=galanin, M617, AR-M) in the presence of M40 (LX-2 cells+(Ch+X+M40)). #: galanin, M617, Ar-M, M871 vs vehicle. N=4, p<0.05.

14. Discussion

The role of Gal and its receptors GalR1 and GalR2 in modulating biliary hyperplasia and fibrinogenesis was investigated in a model of hepatic cholestasis in vivo, and also by using cholangiocytes and LX-2 cells, in vitro. The Mdr2KO mouse is an established experimental model to study hepatic inflammation and cholestasis, and it is widely used to investigate the initiation and progression of cholestasis (Mauad et al. (1994) Am. J. Pathol. 145: 1237-45; Trauner et al. (2007) Semin. Liver Dis. 27: 77-98). First, the time-course of Gal mRNA expression in the liver of Mdr2KO mice as compared to normal, FVBN controls, was examined. It was found that: i) there was a gradual increase in Gal expression in the liver over time from 1 week to 2 months old mice, either FVBN or Mdr2KO mice; this increase was followed by a decline in 4 months old FVBN and Mdr2KO mice; ii) at all tested timepoints, the expression of Gal in the liver, was significantly greater in Mdr2KO mice than in FVBN controls. When the time-course of CK19 expression was tested in the liver of Mdr2KO mice as compared to FVBN controls, a similar expression pattern of this cholangiocyte marker was found. Regression correlation plotting of these data demonstrated a strong correlation between Gal expression and a cholangiocyte marker, suggesting that Gal has a role in the neuroendocrine regulation of cholangiocyte proliferation in hepatic cholestasis. Without wishing to be bound by theory, these results demonstrate that Gal induces cholangiocyte proliferation in bile-duct ligated rats. Next, the effect of exogenous Gal on biliary hyperplasia and hepatic fibrosis in 2 months old, male and female Mdr2KO mice as compared to FVBN controls was examined. The results showed that Gal treatment enhanced cholangiocyte proliferation and fibrogenesis in both FVBN and Mdr2KO mice, with a larger effect in FVBN mice than in Mdr2KO mice. In Mdr2KO mice which expressed an abnormally high level of Gal, the treatment with exogenous Gal added only a little increase in cholangiocyte proliferation and fibrogenesis. However, when Gal receptors GalR1 and GalR2 were blocked with specific vivo morpholino sequences or antagonists, a robust reduction of biliary hyperplasia was observed in Mdr2KO mice. Thus, GalR1 suppression with GalR1-specific vivo morpholino sequences, strongly inhibited cholangiocyte proliferation, while only slightly reducing fibrogenesis markers. A GalR2-specific antagonist M871, caused a significant reduction in the expression of hepatic fibrosis markers such as αSMA, collagen type I and III, while having no effect on cholangiocyte proliferation, in Mdr2KO mice. A pan-antagonist, M40 was able to inhibit both biliary hyperplasia and fibrosis in Mdr2KO mice. These results suggest that Gal has multiple functions in the liver, acting through GalR1 and GalR2 receptors, which mediate different signaling pathways in cholangiocytes and HSC.

The distribution of Gal in various types of cells in the liver of FVBN and Mdr2KO mice, has been also investigated in this study. By using LCM, Gal mRNA was detected in cholangiocytes but not in HSC or hepatocytes collected from livers of FVBN and Mdr2KO mice, suggesting that cholangiocytes are able to synthetize Gal locally, besides the sympathetic nerves in the liver. These results are consistent with published data showing that the liver releases significant amounts of the peptide during sympathetic activation (Kowalyk et al. (1992)Am. J. Physiol. 262: E671-8). Several studies demonstrated that cholangiocytes acquire neuroendocrine features in hepatic cholestasis and other liver diseases (Alvaro et al. (2007) Gastroenterology 132: 415-31). During pathologies related to liver injuries, cholangiocytes start proliferating in an atypical manner and express neuroendocrine genes such as serotonin (Marzioni et al. (2005) Gastroenterology 128: 121-37), endogenous opioid peptides (Marzioni et al. (2006) Gastroenterology 130: 1831-47), and somatostatin (Tietz et al. (1995) Am. J. Physiol. 269: G110-8). Without wishing to be bound by theory, the present results indicate that Gal is also produced in cholangiocytes of Mdr2KO mice, and are in line with previous data from bile duct ligated rats (McMillin et al. (2017) Am. J. Pathol. 187: 819-830). The presence of Gal peptide in various types of cells in the liver was also assessed by confocal microscopy and co-localization with known markers of cholangiocytes, HSC and hepatocytes. The intracellular distribution of Gal was different in cholangiocytes as compared to HSC and hepatocytes—there was a large amount of Gal inside cholangiocytes, in the cytoplasm, while smaller amounts of Gal were associated with HSC and hepatocytes, mostly around their plasma membranes.

The cellular distribution of Gal receptors GalR1 and GalR2 in the liver, in Mdr2KO and FVBN mice, was studied using LCM. Cholangiocytes of FVBN and Mdr2KO mice, expressed almost exclusively GalR1, and these data confirmed previously published results from bile duct ligated rats (McMillin et al. (2017) Am. J. Pathol. 187: 819-830). In contrast, the HSC and hepatocytes expressed mainly GalR2. Interestingly, the LCM results indicated that GalR1 mRNA was greater in cholangiocytes from Mdr2KO mice as compared to cholangiocytes from FVBN control mice, which is consistent with a larger IBDM in Mdr2KO mice vs FVBN mice.

A possible coordination of GalR1 and GalR2 activation in cholangiocytes and HSCs, in the process of Gal-stimulated fibrogenesis, was investigated by in vitro experiments using mouse pooled cholangiocytes and LX-2 cells in culture. Without wishing to be bound by theory, the results suggest that: i) cholangiocytes were stimulated by Gal and GalR1-specific agonist M617 to produce conditioned media which was able to induce activation of LX-2 cells and GalR1 was critical in this mediation; ii) LX-2 cells were activated and expressed fibrotic markers such as αSMA or Col1A1 when treated with Gal or GalR2-specific agonist AR-M but not by GalR1-specific agonist M617; iii) LX-2 cells were activated by conditioned media of cholangiocytes treated with Gal or GalR1-specific agonist but not with GalR2-agonist; iv) GalR1-siRNA knockdown in cholangiocytes prevented activation of LX-2 cells when cholangiocytes were stimulated with Gal or M617 GalR1-agonist; v) GalR2 inhibition by M871 antagonist in LX-2 cells resulted in no activation induced by Gal or cholangiocyte-conditioned media when cholangiocytes were stimulated with Gal; and vi) LX-2 cells were highly activated by Gal when treated with media from cholangiocytes with GalR2-antagonist.

Thus, Gal stimulates proliferation of cholangiocytes via GalR1 and enhances HSC activation via GalR2. It was previously determined that in cholangiocytes, Gal induced cell proliferation by a signal transduction pathway involving extracellular signal regulated kinase (ERK) 1/2, ribosomal S6 kinase 1 and cAMP responsive element binding protein (CREB) activation (McMillin et al. (2017)Am. J. Pathol. 187: 819-830). The molecular mechanisms of Gal-induced activation of HSCs are still to be explored. Findings related to GalR2 being the main Gal receptor expressed in HSC in vivo are in agreement with a study made by H E et al. using HSC T6 cells line in vitro (He et al. (2016) Exp. Ther. Med. 12: 3375-3380); however, unlike the results obtained with T6 cell line, these data, which were taken from both in vivo and in vitro experiments, indicate that GalR2 in HSC contributes to Gal-induced fibrogenesis.

Data from in vivo and in vitro experiments suggest that Gal, which is produced in the liver initially by sympathetic nerves upon stimulation, signals cholangiocytes to produce and secrete more Gal, with autocrine and paracrine effects, depending on the nature of Gal receptors of the surrounding cells. Thus, cholangiocytes treated with GalR1-specific agonists are able to condition their culture medium so that it induces activation of HSC via GalR2. Herein, it is demonstrated that functional GalR1 in cholangiocytes is essential for an indirect activation of HSC via cholangiocyte-conditioned culture medium. GalR2 in the HSC is also critical, and GalR2 has to be functional in order for HSC to be activated via cholangiocyte-conditioned medium. The biosynthesis of Gal in cholangiocytes amplifies the signal from sympathetic nerves. In hepatic cholestasis, it is demonstrated herein that Gal is abnormally increased in the liver of Mdr2KO mice as compared to FVBN controls. One mechanism by which galanin is increased in cholestatic mice, may be explained by an enhanced pituitary secretion of Gal in response to strong reduction of corticosterone caused by hepatic fibrosis. It was previously demonstrated that in Mdr2KO mice, the hypothalamus-pituitary-adrenal (HPA) axis was suppressed resulting in lower than normal levels of corticosterone (Petrescu et al. (2017) Int. J. Mol. Sci. 18). According to other publications, in adrenalectomized rats, Gal-immunoreactive nerve fibers in the anterior pituitary gland were increased and presented more frequent ramifications (Liu and Ju (1998) Acta Histochem. 100: 149-56). It was also demonstrated that Gal, as a neuropeptide involved in the regulation of growth axis, is negatively modulated by glucocorticoids (GC) (Brogan et al. (1999) Metabolism 48: 792-6; Giustina et al. (1995) Metabolism 44: 224-7). Thus, dexamethasone significantly decreased somatic growth of male adult rats, while it decreased Gal mRNA level in hypothalamus and pituitary gland, suggesting a crosstalk and opposite actions of Gal and GC in regulating pituitary growth hormone (GH) production (Brogan et al. (1999) Metabolism 48: 792-6). In contrast, Gal was shown to stimulate GC production. Several studies on the molecular mechanisms underlying the neuroendocrine regulation of the HPA axis, demonstrated that Gal stimulated GC secretion in vivo through a receptor-dependent activation of the adenylate cyclase (AC)/protein kinase A (PKA)-mediated pathway (Mazzocchi et al. (1998) Peptides 19: 891-5). Studies on rat inner adrenocortical cells, which were shown to express GalR1 and GalR2 but not GalR3 receptor, indicated that Gal stimulated corticosterone secretion via these two receptor subtypes which were coupled to the AC/PKA-dependent pathway (Andreis et al. (2007) Int. J. Mol. Med. 19: 149-55; Belloni et al. (2007) Int. J. Mol. Med. 20: 859-64). In the case of cholestatic disease, however, in spite of increased Gal, there is an impairment of GC production in the adrenals due to multiple and complex factors involved in the disease, including the dysregulation of HPA axis by bile acids (Quinn et al. (2014) Dig. Liver Dis. 46: 527-34; McMillin et al. (2015) Mol. Endocrinol. 29: 1720-30; McNeilly et al. (2010) J. Hepatol. 52: 705-11). In a normal, healthy liver, a finely tuned balance between neural/pituitary Gal secretion and GC production in the adrenals, maintains the optimal neuroendocrine homeostasis. However, in the cholestatic liver, while Gal is produced in excess to compensate for the lack of GC, side effects of excessive Gal affect the liver in a negative way. Excessive and chronic Gal secretion results in increased hepatic Gal, causing cholangiocyte proliferation, activation of HSCs, and increased hepatic fibrogenesis. In conclusion, the present study demonstrates that inhibitors of GalR1 and GalR2 receptors reduced cholangiocyte proliferation and hepatic fibrosis markers in Mdr2KO mice and, thus, may be valuable in treating and managing hepatic cholestasis and cirrhosis.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating liver fibrosis in a subject, the method comprising administering to the subject an effective amount of at least one agent that modulates galanin receptor 1 (GalR1) and galanin receptor 2 (GalR2), or a pharmaceutically acceptable salt thereof.

2. The method of claim 1, wherein the subject is a mammal.

3. The method of claim 1, wherein the subject is a human.

4. The method of claim 1, wherein the subject has been diagnosed with a need for treatment of liver fibrosis prior to the administering step.

5. The method of claim 1, wherein the subject is at risk for developing liver fibrosis prior to the administering step.

6. The method of claim 1, further comprising the step of identifying a subject in need of treatment of liver fibrosis.

7. The method of claim 1, wherein the agent that modulates GalR1 and GalR2 is an antagonist of GalR1 and GalR2.

8. The method of claim 1, wherein the agent that modulates GalR1 and GalR2 is M40.

9. The method of claim 1, wherein the effective amount is a therapeutically effective amount.

10. The method of claim 1, wherein the effective amount is a prophylactically effective amount.

11. A method for treating a fibrotic disorder in a subject, the method comprising co-administering to the subject an effective amount of at least one agent that modulates GalR1, or a pharmaceutically acceptable salt thereof, and at least one agent that modulates GalR2, or a pharmaceutically acceptable salt thereof.

12. The method of claim 11, wherein the agent that modulates GalR1 is a GalR1 antagonist.

13. The method of claim 12, wherein the GalR1 antagonist is a vivo-morpholino sequence or a GalR1-specific siRNA.

14. The method of claim 11, wherein the agent that modulates GalR2 is a GalR2 antagonist.

15. The method of claim 14, wherein the GalR2 antagonist is M871.

16. The method of claim 11, wherein the at least one agent that modulates GalR1 is a GalR1 antagonist and wherein the at least one agent that modulates GalR2 is a GalR2 antagonist.

17. The method of claim 11, wherein the effective amount is an individually effective amount of the agent that modulates GalR1 or the agent that modulates GalR2.

18. The method of claim 11, wherein the effective amount is a combinatorically effective amount of the agent that modulates GalR1 and the agent that modulates GalR2.

19. The method of claim 11, wherein the fibrotic disorder is liver fibrosis.

20. A pharmaceutical composition comprising:
a) at least one agent that modulates GalR1, or a pharmaceutically acceptable salt thereof;
b) at least one agent that modulates GalR2, or a pharmaceutically acceptable salt thereof; and
c) a pharmaceutically acceptable carrier,
wherein at least one of the agent that modulates GalR1 and the agent that modulates GalR2 is present in an effective amount.
